(19) United States
(12) Patent Application Publication
(10) Pub. No.: US 2026/0369417 A1
(43) Pub. Date: Nov. 19, 2026

(54) COMPUTER-BASED SYSTEMS AND METHODS FOR EARLY DETECTION OF PATIENT HEALTH DETERIORATION

(71) Applicant: THE FEINSTEIN INSTITUTES FOR MEDICAL RESEARCH, INC., Manhasset, NY (US)

(72) Inventors: Theodoros ZANOS, Manhasset, NY (US); Michael SCHEID, Manhasset, NY (US)

(21) Appl. No.: 19/655,098

(22) Filed: Apr. 22, 2026

Related U.S. Application Data
(60) Provisional application No. 63/793,158, filed on Apr. 23, 2025.

(51) Int. Cl. G16H 50/30; G16H 10/60; G16H 40/67; G16H 50/20

(52) U.S. Cl. CPC G16H 50/30; G16H 10/60; G16H 40/67; G16H 50/20

(57) ABSTRACT

A system including a wearable device patched to a body of a person for measuring a vital sign thereof, a controller configured to schedule the wearable device to continuously measure the vital sign of the person at a predetermined interval to generate a stream of vital data, a signal processing engine configured to receive the stream of vital data from the wearable device and measurement data from an electronic medical record associated with the person, the measurement data being time overlapped with the stream of vital data, the signal processing engine configured to validate the stream of vital data by comparing the stream of vital data with the measurement data, a trained model configured to receive the validated stream of vital data to generate a stream of inferences, and an alert generator configured to generate an alert based on comparing the stream of inferences with a predetermined rule.

### CROSS REFERENCE TO RELATED APPLICATION

This application claims the benefit or priority to U.S. Provisional Application No. 63/793,158, filed Apr. 23, 2025, entitled “SYSTEMS AND METHODS FOR EARLY DETECTION OF PATIENT HEALTH DETERIORATION”, the specification and drawings of which are incorporated herein by reference.

### GOVERNMENT SUPPORT

This invention was made with government support under NR020774 awarded by the National Institutes of Health. The government has certain rights in the invention.

### FIELD OF TECHNOLOGY

The present disclosure generally relates to the field of patient health monitoring, and, more particularly, to early detection of patient health deterioration.

### BACKGROUND OF TECHNOLOGY

Typically, up to 5% of patients admitted to non-critical care inpatient units develop deterioration requiring escalation of care and transfer to a critical care setting. Typically, delayed recognition of a critical illness is associated with increased morbidity and mortality. Since a typical standard of non-intensive care unit (ICU) care may rely on intermittent vital signs measured every four to eight hours, the intervening period between measurements can result in missed warning signs of deterioration.

A typical conventional approach to patient monitoring on medical units all around the country is quite labor intensive and infrequent, every 4 to 8 hours or longer. In those hours, a lot of things can go wrong. Typically, subtle changes in vitals may go undetected, leading to delayed interventions, costly ICU transfers, extended lengths of stay and poorer patient outcomes. For example, in some health care systems, whin a single hospital, if 10% of patients deteriorate, such scenario may result in many tens of thousands deterioration events per year. Across the country, a number of deteriorations may grow to several millions deteriorations annually. Typically, a single deterioration may cost at least several tens of thousands dollar which may result in tens of billions in healthcare costs annually.

### SUMMARY OF DESCRIBED SUBJECT MATTER

The present disclosure is directed to exemplary computer systems, computing devices, and software for efficient patient monitoring on the medical-surgical wards. Such monitoring is crucial to prevent significant in-hospital adverse events. Standard episodic inpatient assessment of vital signs can potentially miss changes in health status and delay recognition of elevated risk. To reduce the likelihood of this delayed recognition of risk, a wearable-based deep learning model is developed, using, for example, 9 inputs, to identify the onset of deterioration earlier than traditional early warning systems. This model can be generalized to produce clinical alerts ahead of a broad class of significant adverse clinical outcomes, including rapid response team (RRT) interventions, unplanned intensive care unit (ICU) transfers, intubations, cardiac arrests, and in-hospital deaths.

In at least some embodiments, the techniques described herein relate to a system configured for early-detection of an event of health condition deterioration of a subject, including: at least one first wearable device patched to a body of a person for measuring at least one vital sign thereof, where the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof; a controller configured to schedule the at least one first wearable device to continuously measure the at least one vital sign of the person at a first predetermined interval to generate a first stream of vital data; a signal processing engine configured to receive the first stream of vital data from the at least one first wearable device and measurement data from an electronic medical record associated with the person, the measurement data being time overlapped with the first stream of vital data, the signal processing engine configured to validate the first stream of vital data by comparing the first stream of vital data with the measurement data; a trained model configured to receive the validated first stream of vital data to generate a stream of inferences, where the trained model is trained with continuous monitoring data generated by a plurality of wearable devices on multiple persons, and includes a recurrent neural network (RNN) with long-short-term-memory (LSTM) units; and an alert generator configured to generate an alert signal, to a monitoring system associated with the person, based at least in part on comparing the stream of inferences to a predetermined rule, the predetermined rule including a threshold of a modified early warning score (MEWS) and a duration of the stream of inferences being above the threshold.

In at least some embodiments, the techniques described herein relate to a system, where the measurement data are generated by episodically measuring vital signs of the person using standard hospital equipment.

In at least some embodiments, the techniques described herein relate to a system, where the measurement data is selected from systolic blood pressure (SBP), heart rate (HR), respiratory rate (RR), temperature (Temp), oxygen saturation (SpO2), level of consciousness (AVPU) and any combination thereof.

In at least some embodiments, the techniques described herein relate to a system, where the signal processing engine is further configured to reject at least one artifact from the first stream of vital data for validating the first stream of vital data.

In at least some embodiments, the techniques described herein relate to a system, where the at least one artifact includes data assumed of physiologically implausible values.

In at least some embodiments, the techniques described herein relate to a system, further including at least one second wearable device controlled by the controller to continuously measure the at least one vital sign of the person at a second predetermined interval to generate a second stream of vital data.

In at least some embodiments, the techniques described herein relate to a system, where the signal processing engine is configured to receive the second stream of vital data and update the first stream of vital data by comparing the first and second stream of vital data.

In at least some embodiments, the techniques described herein relate to a system, where the first predetermined interval is 2 to 10 seconds and the second predetermined interval is in a range between 1 to 10 minutes.

In at least some embodiments, the techniques described herein relate to a system, where the first stream of vital data is down-sampled to a third predetermined interval and the second stream of vital data is up-sampled to the third predetermined interval.

In at least some embodiments, the techniques described herein relate to a system, where the alert generator is configured to detect a trend in the stream of inferences as a base for generating the alert.

In at least some embodiments, the techniques described herein relate to a system, where the MEWS is calculated from a plurality of parameters including systolic blood pressure (SBP), heart rate (HR), respiratory rate (RR), temperature (Temp), level of consciousness (AVPU), age, and body mass index (BMI).

In at least some embodiments, the techniques described herein relate to a system, where the MEWS is calculated by assigning a point value to each of the plurality of parameters, and the MEWS is a sum of all the point values.

In at least some embodiments, the techniques described herein relate to a system, where the trained model is trained with historical vital data, measurement data and modified early warning scores from multiple persons.

In at least some embodiments, the techniques described herein relate to a system, where the trained model is trained with training samples and labels, the training samples being built as a first and second fixed-length time sequence each containing multiple synchronized features aligned in time between the first and second sequence, the training label being built by comparing a sequence with a predetermined threshold of a modified early warning score (MEWS) and a duration of the sequence being above the predetermined threshold.

In at least some embodiments, the techniques described herein relate to a system, where the trained model is validated with at least one clinical deterioration event including at least one unexpected intensive care unit (ICU) transfer event, intubation event, rapid response team activation event, cardiac arrest event, or in-hospital death event.

In at least some embodiments, the techniques described herein relate to a computer-implemented method, including: receiving, by a signal processing engine, a stream of vital data of a person measured controllably at a predetermined interval by at least one wearable device patched to a body of the person for measuring at least one vital sign thereof, where the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof; receiving, by the signal processing engine, measurement data from an electronic medical record associated with the person, the measurement data being time overlapped with the stream of vital data; validating, by the signal processing engine, the stream of vital data by comparing the stream of vital data with the measurement data; generating, by at least one processor executing a trained model, a stream of inferences from the validated stream of vital data, where the trained model is trained with continuous monitoring data generated by a plurality of wearable devices on multiple persons, and includes a recurrent neural network (RNN) with long-short-term-memory (LSTM) units; and generating, by an alert generator, an alert signal, to a monitoring system associated with the person, based at least in part on comparing the stream of inferences with a predetermined rule, the predetermined rule including a threshold of a modified early warning score (MEWS) and a duration of the stream of inferences being above the threshold.

In at least some embodiments, the techniques described herein relate to a method, where the MEWS is calculated from a plurality of parameters including systolic blood pressure (SBP), heart rate (HR), respiratory rate (RR), temperature (Temp), level of consciousness (AVPU), age, and body mass index (BMI).

In at least some embodiments, the techniques described herein relate to a method, where the MEWS is calculated by assigning a point value to each of the plurality of parameters, and the MEWS is a sum of all the point values.

In at least some embodiments, the techniques described herein relate to a method, where the trained model is trained with historical vital data, measurement data and modified early warning scores from multiple persons.

In at least some embodiments, the techniques described herein relate to a non-transitory computer readable medium storing program instructions that, when executed by a processor, cause a computer system to perform operations including: receiving a stream of vital data of a person measured controllably at a predetermined interval by at least one wearable device patched to a body of the person for measuring at least one vital sign thereof, where the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof; receiving measurement data from an electronic medical record associated with the person, the measurement data being time overlapped with the stream of vital data; validating the stream of vital data by comparing the stream of vital data with the measurement data; generating, by executing a trained model, a stream of inferences from the validated stream of vital data, where the trained model is trained with continuous monitoring data generated by a plurality of wearable devices on multiple persons, and includes a recurrent neural network (RNN) with long-short-term-memory (LSTM) units; and generating an alert signal, to a monitoring system associated with the person, based at least in part on comparing the stream of inferences with a predetermined rule, the predetermined rule including a threshold of a modified early warning score (MEWS) and a duration of the stream of inferences being above the threshold.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a block diagram illustrating a system for early detection of patient health deterioration according to embodiments of the present disclosure.

FIGS. 2A and 2B are exemplary plots illustrating wearable data validation performed by the signal processing engine according to embodiments of the present disclosure.

FIG. 3 is a block diagram illustrating an EHR vs wearable modified early warning score (MEWS) alert analysis according to embodiments of the present disclosure.

FIG. 4A is an example workflow for generating training data and developing a predictive model according to embodiments of the present disclosure.

FIG. 4B illustrates a receiver operating characteristic (ROC) curve for a trained predictive model.

FIG. 4C illustrates a precision-recall curve corresponding to the predictive model of FIG. 4B.

FIGS. 4D and 4E present two performance-evaluation plots for an RNN-based model.

FIG. 5 is a block diagram illustrating data processing for model training according to embodiment of the present disclosure.

FIG. 6 is a flowchart illustrating a process for health deterioration alert generation and training data acquisition according to embodiments of the present disclosure.

FIGS. 7A-7L are Bland-Altman plots illustrating wearable data evaluation according to embodiments of the present disclosure.

FIGS. 7M, 7O and 7P are plots illustrating clinical alert, vital contribution, and movement analysis according to embodiments of the present disclosure.

FIGS. 8A and 8B are plots illustrating retrospective validation on Device #1 data according to embodiments of the present disclosure.

FIGS. 8C and 8D are plots illustrating prospective validation on Device #1 data according to embodiments of the present disclosure.

FIGS. 8E and 8F are plots illustrating alternate device testing on Device #2 according to embodiments of the present disclosure.

FIGS. 8G-8J are plots illustrating lead time and sampling rate analysis according to embodiments of the present disclosure.

FIG. 9 shows an exemplary relationship between signal magnitude area (SMA) and heart rate according to embodiments of the present disclosure.

FIG. 10 shows an exemplary relationship between movement (SMA) and respiratory rate (RR) according to embodiments of the present disclosure.

FIG. 11 shows an exemplary relationship between inter-sampling interval and performance of the wearable based RNN deterioration prediction algorithm according to embodiments of the present disclosure.

FIG. 12 shows sample patient vital signs preceding an ICU transfer even according to embodiments of the present disclosure.

FIG. 13 is a block diagram illustrating an exemplary computer-based system/platform in accordance with one or more embodiments of the present disclosure.

FIG. 14 is a block diagram illustrating a system for early detection of patient health deterioration according to embodiments of the present disclosure.

FIG. 15 is a flowchart illustrating a process for generating health deterioration alert according to embodiments of the present disclosure.

### DETAILED DESCRIPTION

Regarding at least some embodiments of the precent disclosure, to address one technological problem of the delayed intervention in conventional health care systems, an illustrative deterioration prediction AI algorithm and system, the Continuous Clinical Alert System or CCAS for short, may be utilized. In at least some embodiments, the exemplary CCAS used data from clinical wearables to alert clinicians up to 17 hours before a deterioration event, with 95% accuracy. In at least some embodiments, the exemplary CCAS can empower clinicians to intervene early and proactively. In at least some embodiments, in some health care system showed that the exemplary CCAS staved off 35% of the early deterioration in patients.

In at least some embodiments, continuous monitoring (CM) solutions in CCAS can continuously monitor in-patients in places, such as ICUs, step-down units, or telemetry units. A technical problem is that many monitoring systems use simple thresholds or cutoffs that often result in a predictable cascade of events: over-alerting leading to hospital staff alert-fatigue; dismissal of useful alerts; delayed recognition of critical illness; and, ultimately, worse outcomes. In conjunction with more discriminating alerting systems, continuous CM provides a technical solution to the above technical problem and has the potential to facilitate timely and accurate early identification of deterioration on inpatient units.

In at least some embodiments, an exemplary, illustrative, non-limiting, example of using CM devices in accordance with one of more principles of the present disclosure conducted in four hospitals in New York to collect CM data from 888 non-ICU inpatients (i.e., 2,897 patient days). The experiment included validating the correlation between CM and standard vital sign measurements, assessing the feasibility and benefits of using these data to generate clinical alerts, and developing a deep-learning model using CM data to identify patient deterioration up to 24 hours before an EHR-based Modified Early Warning Score (MEWS) clinical alert.

In the illustrative, non-limiting experiment, the CM data were collected using two chest-worn wearable devices and validated the deep-learning model in three stages using three unique datasets: data held out for testing from the device used to train the model (Retrospective Device #1), prospective data from Device #1 at a separate hospital (Prospective Device #1), and data from a second wearable device (Alternate Device #2) used on a different patient population at a different hospital to function as an alternate device validation. To establish both the accuracy and specificity of a real-time implementation of this CM-based inpatient deterioration detection algorithm, the experiment includes simulating deployment of an algorithm by generating a stream of continuous inferences at all timepoints where patients were wearing a wearable device. The experiment included measuring the performance of the deterioration prediction algorithm in detecting deterioration, as well as the rate of false alerts, at all timepoints in which valid CM data was available.

This illustrative, non-limiting experiment employed two different clinical grade wearable biosensors (4-8% data missingness, excluding SpO2), as part of a quality initiative, a recurrent neural network (RNN) was trained to predict both modified early warning score (MEWS) alerts and adverse clinical outcomes. Using multiple stages of validation, it was shown that in a retrospective, time-sequence duration optimized, prospective validation, the RNN model was able to predict both periods of elevated MEWS scores (ROC AUC 0.89+/−0.3, PR AUC 0.58+/−0.14) and adverse clinical outcomes (accuracy: 81.8% on 11 events) up to an average of 17 hours in advance. Our results show that presently disclosed wearable based RNN alert system outperforms traditional episodic clinical support tools in detecting early onset of inpatient deterioration; enabling timely interventions that can improve outcomes and reduce hospital costs for patients in early stages of deterioration.

In at least some embodiments, the CM data were collected using two chest worn wearable devices, Vital Connect Vital Patch (Device #1) and Biobeat Chest Monitor (Alternate Device #2). The episodic data were collected from the enterprise EHR (Sunrise Clinical Manager, Altera [formerly Allscripts]) and included demographics (e.g., age and body mass index [BMI]) as well as manually monitored vital signs (i.e., heart rate [HR], systolic blood pressure [SBP], respiratory rate [RR], skin and body temperature, and oxygen saturation [SpO2]) and the continuously monitored vital signs (i.e., HR, RR, temperature, and SpO2). It should be noted that the wearable device provided both a skin temperature measurement and body measurement, which reflects core body temperature. Upon placement of the patch, the body temperature is calibrated by the nurse. For the wearable data validation, the body temperature measurement from the device was used. Wearable device data was provided monthly by the device manufacturers.

Below table summarizes data sources for each of the continuous monitoring (CM) and demographics variables from the electronic health record (HER).

| | Retrospective | | Alternate | | Prospective | |
|---|---|---|---|---|---|---|
| Data source | Device #1 | | Device #2 | | Device #1 | |
| Heart rate (HR) | CM | | CM | | CM | |
| Respiratory rate (RR) | CM | | CM | | CM | |
| Temperature (Temp) | CM | | CM | | CM | |
| Oxygen saturation (SpO2) | CM | | CM | | N/A | |
| Systolic blood pressure | EHR | | EHR | | EHR | |
| (SBP) | | | | | | |
| Age | EHR | | EHR | | EHR | |
| Body mass index (BMI) | EHR | | EHR | | EHR | |
| Signal magnitude area | CM | | | | N/A | |
| (SMA) | | | | | | |
| Modified Early warning | CM | EHR | CM | EHR | CM | EHR |
| score (MEWS) | | | | | | |

The patching of patients was performed as a quality initiative, which augmented the standard of care for these patients. The patients that were patched were selected based often on the best clinical judgement of the staff in each of the wards where the wearable devices were deployed. In practice, this meant the nurse overseeing the patient would often decide whether to have a wearable device placed on the patient or not based on their assessment of how at risk the patient was for decompensating.

In at least some embodiments, a wearable clinical alert system in a real-world clinical environment was developed and evaluated. The wearable clinical alert system demonstrated a viable clinical alternative for monitoring on the medical wards. Increasing frequency of vital signs can provide an advantage over episodic monitoring. The data acquired by wearable devices combined with a machine learning approach can yield an accurate predictive algorithm.

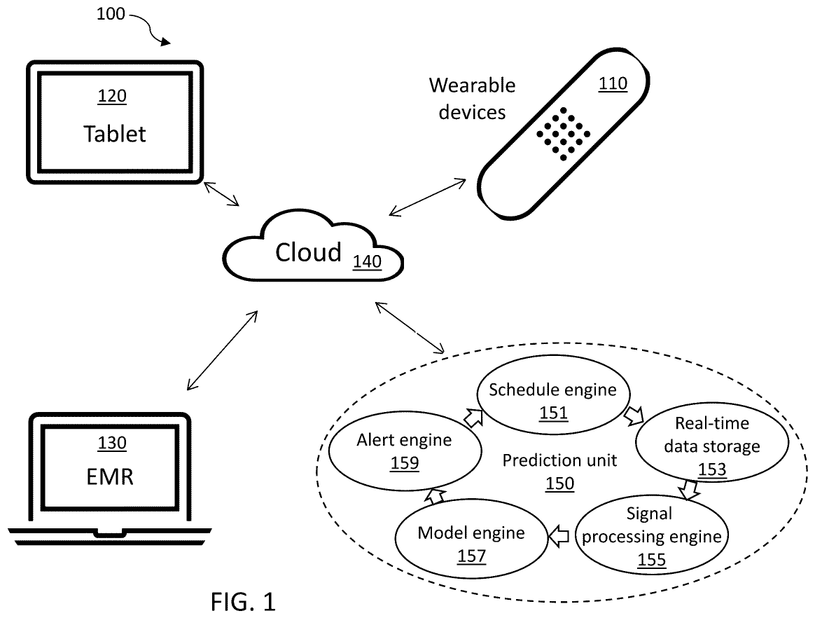

FIG. 1 is a block diagram illustrating a system 100 for early detection of patient health deterioration according to embodiments of the present disclosure. The system 100 includes a plurality of wearable devices 110 patched on a patient's body for continuously measuring vital signs of the patient. Exemplary wearable devices 110 include a Vital Connect vitalpatch, a Biobeat chest monitor, a Masimo radius vital sign monitor (VSM), a biointellisense biobutton, a Neteera 130H-plus, a Sibel health ANNE chest sensor, or an Isansys Lifetouch device. The Vital Connect VitalPatch is a wearable biosensor designed for continuous remote monitoring of vital signs such as heart rate, respiratory rate, skin temperature, and activity level. The Biobeat Chest Monitor is a wearable device designed for continuous, non-invasive monitoring of vital signs such as respiratory rate, blood oxygen saturation, pulse rate, cuff-less blood pressure, and body temperature. The Masimo Radius VSM is a patient-worn, continuous vital signs monitor designed to combine the reliability and accuracy of a bedside monitor with the comfort and freedom of a wearable device. The Masimo Radius VSM can monitor a wide range of physiological measurements, including oxygen saturation (SpO2), pulse rate (PR), perfusion index (Pi), pleth variability index (PVi), plethysmographic respiration rate (RRp), noninvasive blood pressure (NIBP), and skin temperature. The BioIntelliSense BioButton is a medical-grade wearable device designed for continuous remote monitoring of vital signs such as heart rate, respiratory rate, skin temperature, and activity level. The Neteera 130H-Plus is a vital sign monitoring sensor designed for continuous and non-invasive monitoring of various physiological parameters such as heart rate, respiratory rate, and other vital signs continuously. The Sibel Health ANNE Chest Sensor is a clinical-grade wearable device designed for continuous monitoring of various vital signs such as heart rate, respiratory rate, skin temperature, body position, activity, and more. The Isansys LifeTouch device is an advanced wearable biosensor designed for continuous, non-invasive monitoring of vital signs such as heart rate, respiratory rate, heart rate variability (HRV), and provides real-time electrocardiogram (ECG) streaming.

As shown in FIG. 1, the wearable devices 110 may be connected to an exemplary tablet computer 120, an electronic medical record (EMR) 130, and a prediction unit 150 via a computing cloud 140. The tablet computer 120 may provide a user interface for the system 100. A user can access data from the wearable devices 110, the EMR 130 and the prediction system 150 on the tablet computer 120. The user can also enter instructions for controlling the rest of the system 100. The EMR 130 is a digital version of a patient's paper chart, containing medical and treatment history from a specific practice. The EMR 130 may be a part of broader Electronic Health Records (EHRs), which include comprehensive health information and can be shared across different healthcare settings.

As shown in FIG. 1, the prediction unit 150 can make early detection of patient health deterioration based on the continuously measured vital signs of the patient from the wearable devices 110 and corresponding information from the EMR 130 according to embodiments of the present disclosure. In at least some embodiments, the prediction unit 150 may include a schedule engine 151, a real-time data storage 153, a signal processing engine 155, a model engine and an alert engine 159. The schedule 151 controls the wearable devices to continuously measure and collect vital signs. The term, “continuously”, refers generally to repeating measurements in predetermined short intervals, such as in seconds or minutes depends on the capabilities of individual wearable devices. The measured vital signs are then stored in the real-time data storage 153 and passed on to the signal processing engine 155. The signal processing engine 155 may filter noises from and detect trends in the measured vital signs. In at least some embodiments, the signal processing engine 155 implemented artifact rejection to remove segments of data where the measurements assumed physiologically implausible values either due to lost connectivity or low battery issues with the device (i.e., HR or RR were zero and the temperature was above 50 °C). For the purposes of predicting 0.5 to 24 hours into the future, sufficient data of 5.5 hours (i.e., 5-hour sequence+0.5-hour censor) preceding the clinical alert label were needed. We removed the segments and labels where <50% of data was present within the 5-hour sequence.

In at least some embodiments, two devices, Device #1 and Device #2, were used to continuously monitor vital signs. The two devices had different sampling rates and were therefore processed differently to make them compatible with the same deep learning model. Device #1 had a four-second sampling interval and was down sampled to a one-minute interval using a one-minute-wide rolling mean window with a one-minute stride. Alternate Device #2 was sampled variably with a one- to fifteen-minute sampling interval and was up-sampled to one minute using linear interpolation. The EHR data was sampled with a three-to five-hour sampling interval and was up-sampled to one minute using forward-filling imputation. We z-scored the input features to bring them to the same scale for faster model convergence. Whenever continuous data was missing, we used 30 minutes forward filling and imputed with zeros beyond 30 minutes.

As shown in FIG. 1, the processed vital sign data will then be provided to the model engine 157 which is, for example, a trained machine learning model that can make inferences based on the input data. In at least some embodiments, new, unseen data is processed in the same way as the training data. This ensures consistency in how the model interprets the data. The model engine 157 uses the learned patterns to make predictions and generate an output based on the predictions.

As shown in FIG. 1, the alert engine 159 may generate an early detection of the patient health deterioration based on the inferences of the model engine 157, data from the EMR 130 and a predetermined set of rules.

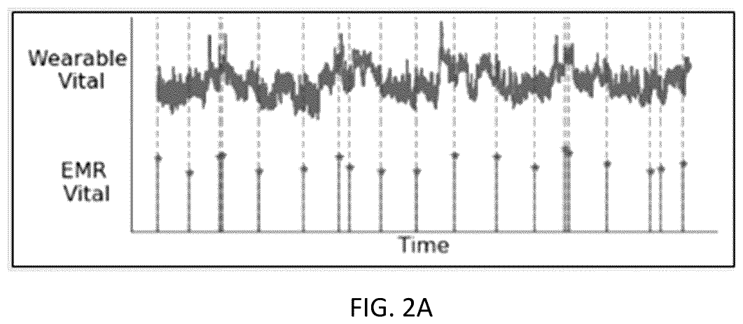

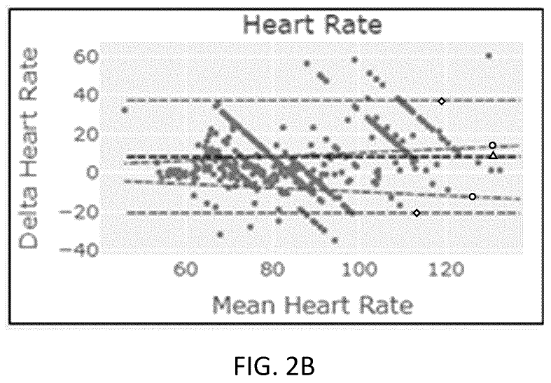

FIGS. 2A and 2B are exemplary plots illustrating wearable data validation performed by the signal processing engine 155 according to embodiments of the present disclosure. Wearable data accuracy validation ensures the data is of sufficient accuracy to use for clinical decision making and this to end, experiments were conducted that include time alignment (FIG. 2A) and visualize differences (FIG. 2B) using Bland-Altman plots, which visualize the difference between the two modalities (y-axis) vs their mean (x-axis).

Referring to FIG. 2A, vital signs measured by the wearable devices 110 (wearable vitals) and vital data in EMR 130 (EMR vitals) are aligned in time. The wearable vitals are continuously monitored (CM) by the wearable devices 110. The EMR vitals are manually acquired and stored in the EMR 130. To synchronize the two data sources, the wearable data was converted from UNIX time to human-readable time, shifted to account for both daylight-savings and time zone differences and then resampled to align to EMR data timestamps. The EMR data was organized chronologically by patient ID number and timestamps and resampled to align to the closest minute with the wearable data.

The correlation between continuous and manually acquired vital signs was examined in order to validate the accuracy and integrity of the clinical wearables themselves and the vital sign measurements made by them. Using this process, the use of one of the vital signs (blood pressure) from the Biobeat device was eliminated in the experiments. The rest of the vital signs measured by the wearables were sufficiently close to the gold standard of the manually measured vital signs so as to make them usable in the deterioration prediction algorithm.

Referring to FIG. 2B, an exemplary Bland-Altman plot is constructed using the time aligned data shown in FIG. 2A. The Bland-Altman plot visualizes the agreement between two measurement modalities by plotting the difference (i.e., Δ) between the time-aligned signal from the two modalities (A=Signal 1 (Wearable)−Signal 2 (EMR)) on the y-axis against their mean (μ=((Signal 1+Signal 2))/2) on the x-axis. The greater the agreement between the two modalities, the closer each of the points on the plot is to the horizontal line, where Δ=0.

For visualization purposes, FIG. 2B plots the 95% confidence intervals (i.e., diamond dotted lines), mean delta (i.e., triangle dotted line) and either the 5% or 10% error line (Δ=μ*0.1) for each of the Bland-Altman plots. The percentage of measurements that fell within the 5% or 10% error line were also calculated. In subsequent analyses, only records where at least ⅔ (i.e., 67%) of a value for a given patient were within a 10% error for HR and a 5% error for temperature and SpO2 from the EMR values were used. This criterion was not applied to respiratory rate because of the well-documented digit preference among healthcare workers when manually measuring the respiratory rate.

In experiments, the above data processing, analysis, and modeling were performed in Python using the Pandas, Numpy, Scikit-learn, Tensorflow, and Keras packages. Visualizations were created using the Matplotlib and Plotly packages.

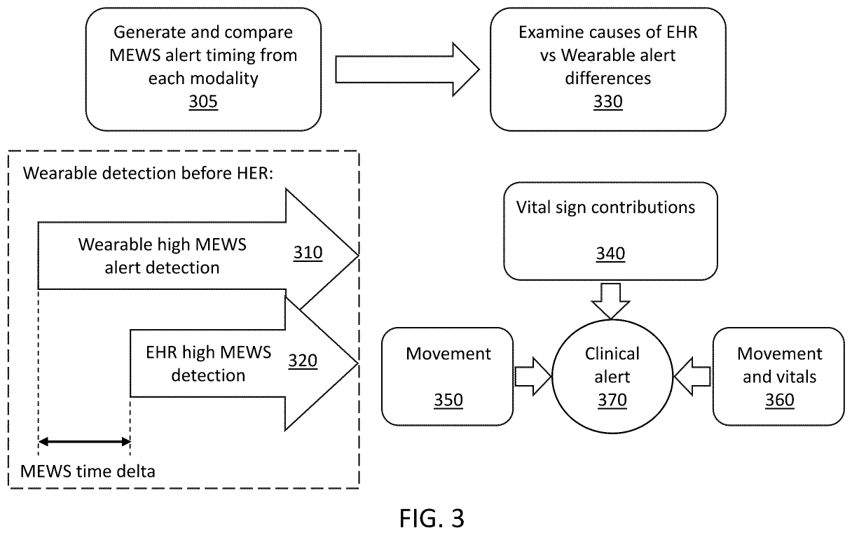

FIG. 3 is a block diagram illustrating an EHR vs wearable MEWS alert analysis according to embodiments of the present disclosure. Generation and comparison of clinical MEWS alerts across measurement modalities in block 305 demonstrates whether a wearable device provides an advantage over episodic monitoring, where experiments were conducted to compare the numbers and timing of alerts for each modality and calculated the sources of these differences.

As shown in FIG. 3, advancement time of high MEWS alerts obtained from a wearable CM system (block 310) and an EHR system (block 320) is compared and a time difference (delta) is calculated. In block 330, causes of the time difference is evaluated. Particular vital sign(s) may be identified as major contributing factor(s) to the time difference (block 340).

As shown in FIG. 3, patient's movement consideration (block 350), the contributing vital signs (block 340), and combined movement-and-vital-sign considerations (block 360) are collectively evaluated to generate a clinical alert in block 370.

To assess the benefits of using wearable data to produce more and earlier clinical alerts, experiments were carried out and compared the volume, timing, and duration of alerts that arose from continuous versus episodic monitoring. The MEWS score were used as the basis for the clinical alerts for a number of reasons: it is derived from measurements acquired across all inpatients; includes a number of vitals that are measured by the wearable device; and is already implemented as part of the standard of care in many hospitals and health systems, including Northwell Health, where it is calculated automatically within the EHR and clinicians are alerted when defined thresholds are exceeded (referring to TABLE 3 below).

The present disclosure details the use of two CM devices in four of Northwell health system hospitals and collected CM data between March 2020 and November 2022 from 888 non-ICU inpatients over 2,897 patient days. TABLE 1 summarizes the demographics of the study population.

TABLE 1
Summarized demographics and statistics across all prediction cohorts.

| | | | | | Alternate | |
|---|---|---|---|---|---|---|
| | Total | | | Retrospective | device | Prospective |
| | All | Stable | Deteriorated | All | All | All |
| Patients (n) | 888 | 871 | 17 | 315 | 413 | 160 |
| Devices (n) | 1302 | 1244 | 58 | 496 | 576 | 230 |
| Male (%) | 453 (51.2) | 445 (51.3) | 8.0 (53.3) | 162 (51.4) | 206 (49.9) | 85 (53.1) |

Race/Ethnicities, n (%)

| | | | | | | |
|---|---|---|---|---|---|---|
| White | 648 (73.3) | 634 (73.1) | 14.0 (93.3) | 214 (67.9) | 328 (79.4) | 106 (66.2) |
| Multiracial | 94 (10.6) | 92 (10.6) | 2.0 (13.3) | 34 (10.8) | 36 (8.7) | 24 (15.0) |
| Black | 86 (9.7) | 85 (9.8) | 1.0 (6.7) | 32 (10.2) | 30 (7.3) | 24 (15.0) |
| Asian | 38 (4.3) | 38 (4.4) | 0 (0.0) | 26 (8.3) | 9 (2.2) | 3 (1.9) |
| Unknown/Other | 16 (2.1) | 18 (2.1) | 0 (0.0) | 9 (2.8) | 6 (1.5) | 6 (2.8) |
| Declined | 4 (0.5) | 4.0 (0.5) | 0 (0.0) | 0 (0.0) | 4 (1.0) | 0 (0.0) |

Model features, median [IQR]

| | | | | | | |
|---|---|---|---|---|---|---|
| Age | 69.3 [57.6, | 69.3 (57.5- | 88.3 (86.5- | 68.0 [56.0, | 70.0 [60.0, | 70.0 [56.8, |
| [median IQR] | 78.0] | 77.3) | 90.5) | 78.0] | 79.0] | 77.0] |
| BMI | 27.0 (23.5- | 27.4 (23.5- | 30.4 [27.3- | 27.4 [23.7, | 28.3 [23.9, | 26.6 [23.0, |
| [median IQR] | 32.3) | 32.3) | 31.8] | 32.1] | 33.3] | 31.5] |
| SMA | 16.3 [8.9- | 16.3 [8.9- | 15.6 [9.1- | 16.3 [8.9- | 0.0 [0.0- | 0.0 [0.0- |
| [median IQR] | 17.7] | 17.7] | 16.3] | 17.7] | 0.0] | 0.0] |
| sBP | 126.0 [109.3- | 125.8 [109.3- | 134.8 [120.3- | 125.2 [110.0- | 127.3 [112.0- | 125.5 [106.0- |
| [median IQR] | 137.7] | 137.7] | 147.8] | 138.0] | 140.0] | 135.0] |
| HR | 79.8 [68.2- | 79.8 [68.1- | 79.8 [70.5- | 79.7 [67.3- | 75.7 [65.4- | 84.0 [71.8- |
| [median IQR] | 90.8] | 90.8] | 90.1] | 90.7] | 84.9] | 96.7] |
| RR | 19.0 [16.5- | 19.0 [16.5- | 19.3 [17.3- | 21.0 [17.4- | 17.6 [16.0- | 18.5 [16.1- |
| (mean SD) | 21.3] | 21.2] | 21.2] | 23.9] | 19.1] | 20.8] |
| Temp | 36.7 [36.3- | 36.7 [36.3- | 36.7 [36.3- | 36.7 [36.3- | 36.6 [36.4- | 36.5 [36.1- |
| (mean SD) | 36.8] | 36.8] | 37.1] | 37.1] | 36.7] | 36.7] |
| SpO2 | 96.0 [95.0- | 96.5 [95.3- | 93.3 [91.1- | 93.0 [90.8- | 96.7 [95.4- | 0.0 [0.0- |
| (mean SD) | 97.9] | 98.2] | 96.3] | 96.0] | 99.0] | 0.0] |
| MEWS | 3.0 [2.0- | 2.9 [2.0- | 5.6 [5.2- | 3.1 [2.2- | 3.0 [2.0- | 2.8 [1.8- |
| (mean SD) | 4.0] | 3.9] | 6.0] | 4.0] | 4.0] | 4.0] |

Abbreviations: BMI=body mass index; HR=heart rate; IQR=interquartile range; Modified Early Warning Score (MEWS); RR=respiratory rate; SBP=systolic blood pressure; SD=standard deviation; SMA=signal magnitude area; SpO2=oxygen saturation; Temp=temperature.

TABLE 2
Confusion matrices for each stage of validation.

| A) Device #1 Retrospective | | Predicted Label | | |
|---|---|---|---|---|
| | Threshold = 0.015 | No Alert - 0 | Alert - 1 | |
| True | No Alert - 0 | 12810 | 13387 | Specificity: 0.49 |
| Label | Alert - 1 | 174 | 3356 | Sensitivity: 0.95 |
| | | NPV: 0.99 | PPV: 0.20 | |
| B) Device #1 Prospective | | Predicted Label | | |
| | Threshold = 0.5 | No Alert - 0 | Alert - 1 | |
| True | No Alert - 0 | 2979 | 1802 | Specificity: 0.62 |
| Label | Alert - 1 | 35 | 459 | Sensitivity: 0.93 |
| | | NPV: 0.99 | PPV: 0.20 | |
| C) Alternate Device #2 | | Predicted Label | | |
| | Threshold = 0.9 | No Alert - 0 | Alert - 1 | |
| True | No Alert - 0 | 33609 | 4981 | Specificity: 0.87 |
| Label | Alert - 1 | 278 | 599 | Sensitivity: 0.68 |
| | | NPV: 0.99 | PPV: 0.11 | |

Abbreviations:
NPV = negative predictive value; PPV = positive predictive value.

Confusion matrix for each of the three validation steps (Device #1 retrospective, Device #1 prospective, Device #2 alternate). The operating point was chosen such that the PPV was equal to 0.2. The threshold that achieved this PPV is displayed in the upper left corner of each table section. Summary metrics (i.e., specificity, sensitivity, PPV, and negative predictive value) are listed at the periphery of each table.

In experiments, RNN and LR model accuracy were evaluated on hard outcomes. Using the RNN model trained on detecting>30-minute periods of elevated MEWS, the experiments were conducted to evaluate the model's performance on predicting hard outcomes within 24 hours (TABLE 3). In summary, 50% of RRT calls, 83.6% of unplanned transfers to the ICU, 100% of intubation/mechanical ventilation, 100% of cardiac arrests, and 100% of death were detected using CM data. This performance was superior to that of using solely EHR data or an elevated MEWS as the predictor for a hard outcome.

TABLE 3
Summary of RNN vs. MEWS performances on hard
outcomes using both wearable and EHR data.

| | | | Wearable | |
|---|---|---|---|---|
| | | EHR RNN | MEWS | EHR MEWS |
| Wearable RNN | | accuracy | accuracy | accuracy |
| accuracy | | (Correct/ | (Correct/ | (Correct/ |
| (Correct/Total) | | Total) | Total) | Total) |
| RRT | 50.0 (1/2) | 100.0 (2/2) | 0.0 (0/2) | 0.0 (0/2) |
| ICU Transfer | 83.3 (5/6) | 50.0 (3/6) | 16.7 (1/6) | 0.0 (0/6) |
| Ventilation | 100.0 (1/1) | 100.0 (1/1) | 0.0 (0/1) | 0.0 (0/1) |
| Cardiac Arrest | 100.0 (1/1) | 0.0 (0/1) | 0.0 (0/1) | 0.0 (0/1) |
| Deaths | 100.0 (1/1) | 0.0 (0/1) | 0.0 (0/1) | 0.0 (0/1) |

In the experiments, two distinct sets of MEWS scores were generated-one derived from continuous wearable vitals and the other from episodic vitals-which resulted in two sets of alerts. The MEWS calculation is based upon vital sign measurements given by the equation below:

$$\begin{matrix}MEWS = {SBP}^{Score} + {HR}^{Score} + {RR}^{Score} + {Temp}^{Score} + {AVPU}^{Score} + {Age}^{Score} \\ + {BMI}^{Score}\end{matrix}$$

The MEWS score is agnostic to adverse outcome, it is a general indicator of physiological stability and, conversely, risk of deterioration of a patient.

Depending on the range of each item, it is given a score ranging from 0 to 3 (referring to above TABLE 2), and the final score is tallied (range 0-15), with higher values indicating greater risk of clinical decompensation. It should be noted that this a commonly used score, not only in our system, but in health systems around the world, as an early warning system. Based upon Northwell Health system protocol (above TABLE 3) and expert consensus among our clinical collaborators, a meaningful clinical alert was defined as a MEWS>6 for 30 minutes or more. In the experiment, the total number of alerts, onset timing, and duration for each alert were quantified. To understand the underlying cause of the differences between CM- and EHR-based clinical alerts, the contribution of each vital sign to the MEWS score from each modality at the time of alert onset was measured.

To rule out the possibility that motion-related events caused temporary changes in vital signs and thereby triggered alerts unrelated to early signs of deterioration, the experiment includes analyzing biomarkers with respect to movement during each alert. Movement was captured by the device with a three-axis accelerometer and represented as the signal magnitude area (SMA), the sum of a one-second average of accelerations in all three axes. The alerts and their corresponding average SMA values, ranging from two minutes preceding the alert onset to the end of the alert, were grouped into three classes: “stationary,” “activities of daily living (ADL),” and “strenuous,” based on manufacturer determined SMA cutoff values (referring to above TABLE 4).

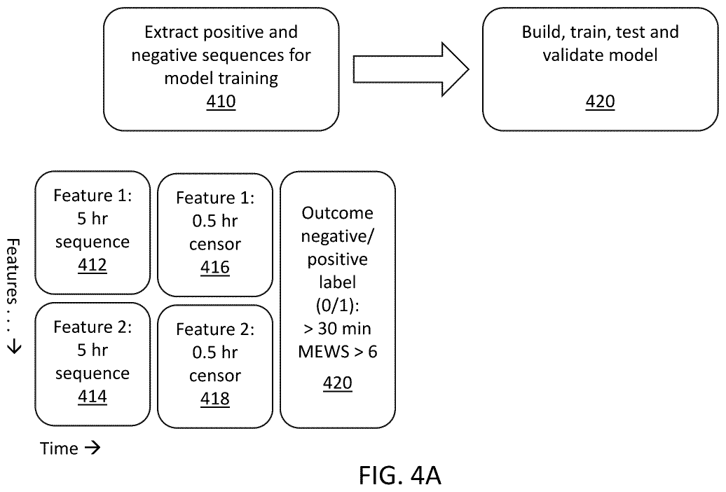

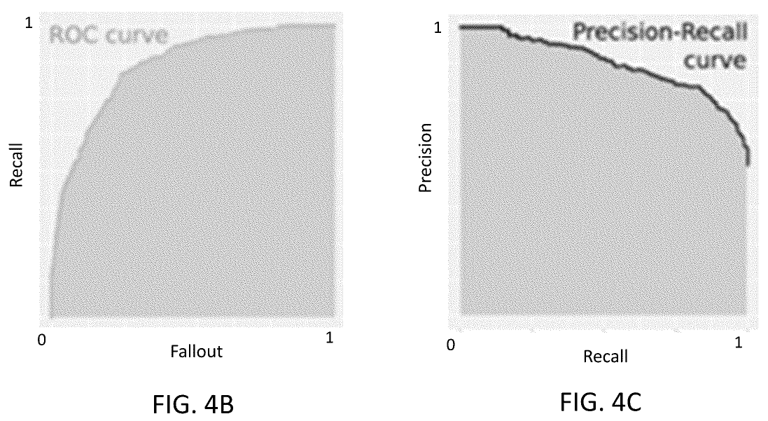

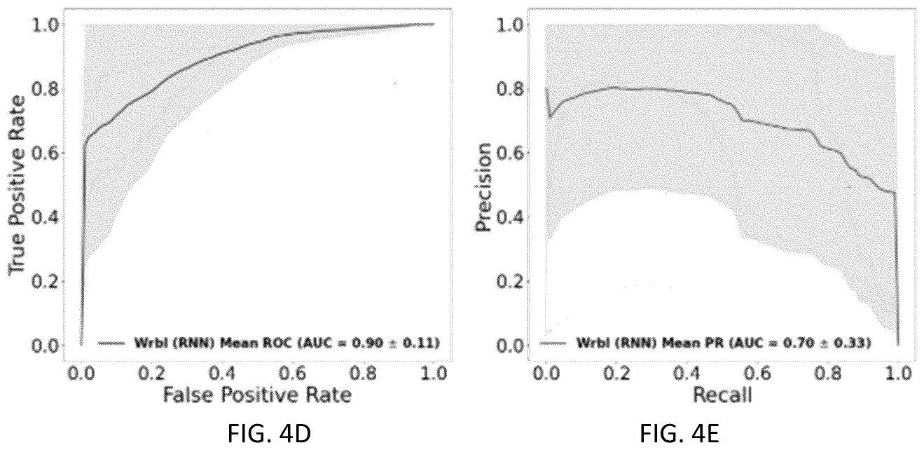

FIG. 4A is an example workflow for generating training data and developing a predictive model according to embodiments of the present disclosure. In at least some embodiments, the clinical alerts were used as labels to train a model and predict periods of clinical deterioration further into the future than current early warning systems. The wearable clinical alert modeling and validation show the feature and label construction and performance of the wearable based RNN deterioration prediction algorithm.

As shown in FIG. 4A, the training process begins with block 410, where positive and negative time-series sequences are extracted for model training. The extracted data include multiple features organized along a time axis, with time progressing horizontally and features arranged vertically.

As shown in FIG. 4A, each feature comprises a defined temporal window of data. For example, Feature 1 and Feature 2 may each include a five-hour sequence of data values (blocks 412 and 414); while corresponding censored features include 0.5-hour segments (blocks 416 and 418) of the same or related signals. Together, these features form a structured input set for training

An outcome label is also associated with each extracted sequence at block 420. The outcome label is binary (0/1) and is determined based on whether a predefined clinical or operational criterion is satisfied. In the illustrated example, the outcome corresponds to an event occurring more than 30 minutes prior to a condition characterized by a Modified Early Warning Score (MEWS) greater than 6. When a sequence produces MEWS>6 for longer than 30 minutes, the sequence is a positive one. A negative sequence include a period with average MEWS<6 and 24 hours from closest alert. Both positive and negative sequences are extracted for training, testing, and validating the model.

Following extraction and labeling of the training sequences, the process advances to block 430, where a model is built, trained, tested, and validated using the extracted positive and negative sequences. The directional arrow between blocks 410 and 430 indicates the flow of data from the feature extraction and labeling stage to the model development stage.

Accordingly, FIG. 4A depicts how temporally aligned multi-feature sequences and associated outcome labels are constructed and then used to train and validate a predictive model.

FIG. 4B illustrates a receiver operating characteristic (ROC) curve for a trained predictive model. The ROC curve plots recall (true positive rate) along the vertical axis against fallout (false positive rate) along the horizontal axis across a range of decision thresholds. The shaded area under the curve represents the overall classification performance of the model, with curve values approaching the upper left corner indicating improved discrimination between positive and negative outcomes. As shown, the curve demonstrates high recall at relatively low fallout levels, indicating effective prediction capability.

FIG. 4C illustrates a precision-recall curve corresponding to the predictive model of FIG. 4B. Precision is plotted along the vertical axis and recall is plotted along the horizontal axis, again across varying decision thresholds. The curve shows how prediction precision decreases as recall increases, reflecting the tradeoff between capturing a higher proportion of true positive events and maintaining predictive accuracy. The shaded region under the curve provides an indication of the model's effectiveness in identifying positive outcomes, particularly in scenarios involving class imbalance.

In-hospital clinical deterioration often precedes adverse outcomes such as unplanned ICU transfer, mechanical ventilation, cardiac arrest, or death, and remains challenging to identify in a timely manner using traditional monitoring approaches. Although electronic health records (EHRs) contain rich longitudinal clinical data, deterioration events are documented across heterogeneous sources and formats, complicating reliable outcome definition and prediction.

This disclosure describes systematic methods for identifying and extracting multiple clinically meaningful deterioration outcomes from EHR data, including unexpected Intensive Care Unit (ICU) transfer events, intubation and extubation events, rapid response team activation events, cardiac arrest events, and expiration events. Using these derived outcomes, a recurrent neural network model incorporating demographic data, intermittent measurements, and continuous physiologic signals is trained to predict near-term deterioration risk, enabling early detection and improved clinical decision support.

In at least some embodiments, the unexpected ICU events are identified from patient electronic health record data, including physician orders, admission records, patient location history, and clinical documentation. Potential ICU transfer events were extracted from these diverse data sources by classifying unit names and narrative content to determine a patient's acuity status (e.g., ICU, stepdown, telemetry, or non-ICU). All identified events were chronologically ordered per patient encounter, and increases in acuity status were flagged as potential deterioration events. These potential deteriorations were subsequently filtered to exclude planned transfers, such as those occurring at or prior to admission, or within a specified time window following surgical procedures, including cases with a documented planned ICU disposition. The final output comprised verified unexpected ICU deterioration events, detailing their acuity level and originating data source.

In at least some embodiments, the onset of mechanical ventilation was determined by identifying intubation events from multiple data sources. Direct indicators included clinical notes categorized as “Airway Placement Note Adult” or “Tracheal Intubation.” Indirect indicators included ventilator orders specifying advanced mechanical ventilation modes, such as “AC/CMV” or “PRVC.”

In at least some embodiments, the extubation events, signifying the cessation of mechanical ventilation, are identified from clinical documentation. This included “Airway Removal Notes” and “Mechanical Vent Records” explicitly indicating that ventilator support was “discontinued.”

All identified intubation and extubation events were chronologically ordered for each patient encounter. A patient's intubation status was tracked, and the precise timestamp of mechanical ventilation onset was defined as the first event marking a transition from an un-intubated to an intubated state.

In at least some embodiments, the expiration or death events are identified from three distinct electronic health record sources. These sources included clinical notes containing keywords indicating “Expired,” patient demographic data where a time of death was recorded within seven days post-discharge and visit discharge dispositions specifying “Expired” or “Coroner.” For each patient encounter, the earliest timestamp across all identified death events was retained.

In at least some embodiments, the rapid response team (RRT) activation events are extracted directly from a dedicated RRT registry, capturing the encounter number and the timestamp of the RRT call.

In at least some embodiments, the cardiac arrest events are identified from a specialized cardiac arrest registry. Events are linked to patient encounters using medical record numbers and validated to ensure the arrest timestamp occurred between the patient's admission and discharge dates for the associated encounter.

In an experiment, the in-hospital deterioration outcomes and their respective frequencies were tallied for a predetermined period of time. A total of 196 deterioration events were observed across all outcome categories. The most common event was unexpected ICU transfer (126 events), which accounted for the majority of deteriorations. This was followed by expiration (28 events), rapid response team (RRT) activations (22 events), and intubation events (19 events). Cardiac arrest was rare, with a single documented event. Collectively, this experiment highlights that most deterioration episodes involved escalation of care rather than terminal events, underscoring the importance of early detection prior to severe clinical decline. The above in-hospital deterioration outcomes are also summarized in below table.

| Outcome | No. of events |
|---|---|
| ICU transfer | 126 |
| Expiration | 28 |
| RRT | 22 |
| Intubation | 19 |
| Cardiac arrest | 1 |
| Total | 196 |

In some embodiments, for early detection of clinical deterioration, a recurrent neural network (RNN) with long-short-term-memory (LSTM) units model is trained that incorporated age, BMI, SBP, and continuously monitored vital signs (HR, RR, temperature, SpO2, MEWS value, motion signals and two measures of heart rate variability (HRV)) to predict in-hospital deterioration with a 0.5-24 hour time horizon using a 5 hour sequence. The deterioration labels were constructed based on the criteria described in FIG. 4A and associated description hereinabove.

The predictive performance of this model was evaluated using 5-fold stratified shuffle split cross-validation at the patient level, i.e., no patients in the training set were included in the test set. This resulted in an approximately 80%/20% train/test split. We then compared predictive performance across all folds, using common model validation metrics (ROC AUC, PR AUC) and averaged the resulting ROC and PR curves.

FIGS. 4D and 4E present two performance-evaluation plots for an RNN-based model. The data used in the plots reflects the classifier performances on an expanded set of 196 hard outcomes (deterioration events). These in-hospital deterioration event outcomes and their respective frequencies were tallied for a predetermined period of time. Among the 196 deterioration events, the most common event was unexpected ICU transfer (126 events), which accounted for the majority of deteriorations. This was followed by expiration (28 events), rapid response team (RRT) activations (22 events), and intubation events (19 events). Cardiac arrest was rare, with a single documented event.

FIG. 4D illustrates a receiver operating characteristics (ROC) curve which shows one of strong separability. As the false positive rate increases, the true positive rate rises quickly and smoothly, with the mean ROC curve bending sharply toward the upper-left corner of the plot. This shape indicates that the model is able to correctly identify the majority of positive cases while keeping false positives relatively low. The high mean AUC of about 0.90 reinforces this observation, suggesting that, overall, the model has excellent discriminative power. The shaded region around the curve reflects variability across runs, but even with this spread, performance remains consistently high across most thresholds.

FIG. 4E illustrates a precision-recall (PR) curve which shifts the focus from overall discrimination to the balance between precision and recall. Here, precision is fairly high at lower recall values, indicating that when the model makes positive predictions conservatively, most of those predictions are correct. As recall increases, precision gradually declines, revealing the trade-off that emerges as the model attempts to capture more truly positive cases. The mean PR AUC of about 0.70 suggests moderate performance in this setting, and the wide shaded band highlights substantial variability between runs. This pattern implies that while the model is capable of identifying many positives, achieving high recall comes at the cost of increased false positives.

Taken together, the FIGS. 4D and 4E suggest that the RNN model is highly effective at ranking and separating classes overall, as demonstrated by the ROC analysis, but its practical performance in terms of precision versus recall depends strongly on the chosen operating point. This underscores the importance of threshold selection, especially in applications where false positives or false negatives carry different consequences.

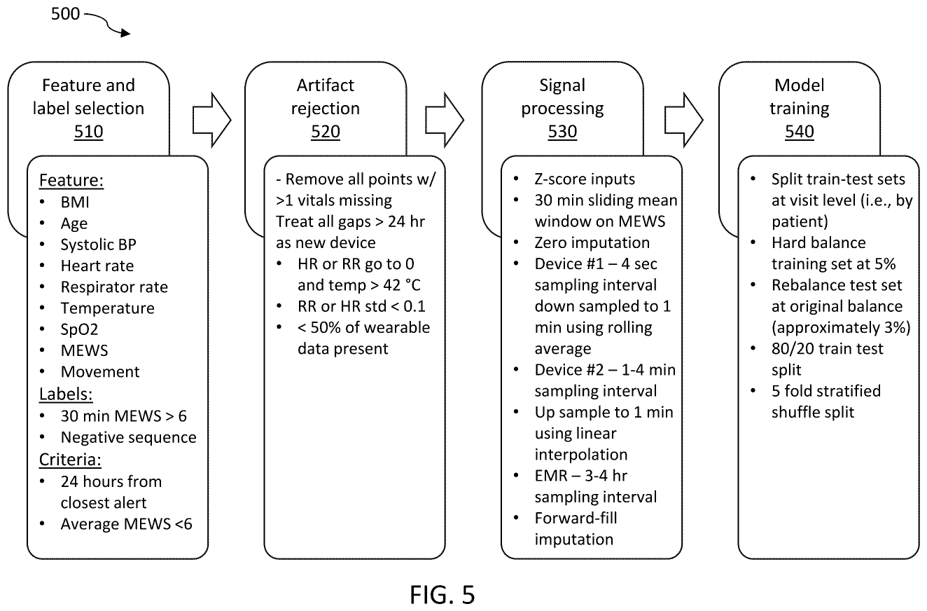

FIG. 5 is a block diagram illustrating data processing 500 for model training according to embodiment of the present disclosure. The data processing 500 exemplarily includes a feature and label section block 510, an artifact rejection block 520, a signal processing block 530 and a model training block 540 as described herein below.

In block 510, following patient's features are exemplarily selected: BMI, age, systolic BP, heart rate, respiratory rate, temperature, SpO2, MEWS, and movement. In addition, following labels are also exemplarily selected: 30 min MEWs>6, negative sequence criteria, 24 hours from closest alert, and average MEWS<6.

In block 520, artifact rejection may be implemented to remove segments of data where the measurements assumed physiologically implausible values either due to lost connectivity or low battery issues with the device (i.e., HR or RR were zero and the temperature was above 50 °C). In order to determine the optimal window length for predicting clinical alerts 0.5 to 24 hours in the future, we performed a grid search of window lengths from 1 to 12 hours at 1-hour increments and determined 5 hours to be the optimal length based on retrospective model performance. For the purposes of predicting 0.5 to 24 hours into the future, sufficient data of 5.5 hours (i.e., 5-hour sequence+0.5-hour censor) preceding the clinical alert label were needed. The segments and labels where <50% of data was present within the 5-hour sequence may be removed.

In block 530, the two devices that were used to continuously monitor vital signs had different sampling rates and were therefore processed differently to make them compatible with the same deep learning model. Device #1 had a four-second sampling interval and was down sampled to a one-minute interval using a one-minute-wide rolling mean window with a one-minute stride. Alternate Device #2 was sampled variably with a one-to fifteen-minute sampling interval and was up-sampled to one minute using linear interpolation. The EHR data was sampled with a three-to five-hour sampling interval and was up-sampled to one minute using forward-filling imputation. We z-scored the input features to bring them to the same scale for faster model convergence. Whenever continuous data was missing, we used 30 minutes forward filling and imputed with zeros beyond 30′. For the prospective Device #1, SpO2 was not available so the variables was imputed with all zeroes for the final model.

In block 540, the process 500 includes training a recurrent neural network (RNN) with long-short-term-memory (LSTM) units model that incorporated age, BMI, SBP, and continuously monitored vital signs (HR, RR, temperature, SpO2, MEWS value, and motion signals) to predict in-hospital deterioration with a 0.5-24 hour time horizon using a 5 hour sequence (FIG. 2). The clinical alert labels detailed in block 510 were constructed based on smoothed MEWS values using a 30-minute-wide rolling average window.

In at least some embodiments, the model is trained to predict a longer than 30-minute period of elevated MEWS (score greater than 6.5 on 15-point scale). The model's ability to predict adverse clinical outcomes is a generalization effect that is likely due to the direct relationship between periods of elevated MEWS and the adverse outcomes on which the model generalizes. Thus, the model is not trained at all on the adverse clinical outcomes and is only trained on the periods of elevated MEWS. The periods of elevated MEWS label is used as a proxy label for physiologic deterioration in order to provide enough labels for model training.

In order to select a model to test on clinical alerts and hard outcomes we performed hyperparameter optimization across the following parameters: number of LSTM layers, sequence length, time horizon, and censor length. The selected model showed superior performance across the various validation steps performed in this study.

Class imbalance was addressed by first randomly removing samples from the negative majority class and oversampling the positive class until we achieve a balance of 95%/5% negative/positive classes of the training set, instead of the original 99.7%/0.3% class balance.

The predictive performance of this model was evaluated in three stages: retrospective, prospective, and alternate Device validations. In the first retrospective validation stage, we validated the model using 5-fold stratified shuffle split cross-validation at the patient level, i.e., no patients in the training set were included in the test set. This resulted in an approximately 80%/20% train/test split. We then compared predictive performance across all folds, using common model validation metrics (ROC AUC, PR AUC) and averaged the resulting ROC and PR curves. After the initial validation stage, we then tested the model in two additional validation stages using two unique datasets: 1) data collected prospectively from Device #1, i.e. entirely after Device #1's retrospective data used to train the model, at a separate hospital and from a different patient cohort, and 2) data from a second wearable device used at a separate hospital and on an entirely different patient population to function as an alternate device test set. In each subsequent stage of validation, i.e., “Prospective” and “Alternate Device” validations, the model that was trained exclusively on Device #1's retrospective dataset was then tested on each of these pure validation datasets. Neither data from prospective Device #1 data nor Alternate Device #2 was used to optimize, hyperparameter tune or train the retrospectively trained model in anyway. These datasets, and their accompanying validation results, therefore functioned as two true unique test sets for the retrospectively optimized and trained model.

These three validation stages contrasted performance of the LSTM RNN model against logistic regression (LR) using two concurrently collected datasets: 1) CM data from the wearable device and, 2) EHR data collected alongside that wearable data (EHR).

In experiments, the wearable RNN model accuracy were evaluated on hard outcomes. Using the RNN model trained to detect>30-minute periods of elevated MEWS, the model's performance on prespecified “hard outcomes” were evaluated with sufficient wearable data prior (i.e., at least 5.5 hours). In total 11 hard outcomes were found in the Retrospective Device #1 dataset with sufficient preceding wearable data to make predictions. It was also ensured that the hard outcomes being predicted upon were at least 24 hours away from each other. To predict hard outcomes, the experiments used the same features, processing steps, and testing procedure of the previous modeling steps with the exception that the label being predicted was one of five hard outcomes: RRT call, unplanned transfer to the ICU, intubation/mechanical ventilation, cardiac arrest, and death (expiration). Successful detections were counted if a true positive was generated within the 24 hours leading up to the outcome. The performance of the RNN algorithm in detecting hard outcomes was compared to using CM data against EHR data alone. A comparison was also made using an elevated MEWS (>6) as the predictor for the hard outcome. In an experiment, the in-hospital deterioration outcomes and their respective frequencies were tallied for a predetermined period of time. A total of 196 deterioration events were all observed across all outcome categories. The most common event was unexpected ICU transfer (126 events), which accounted for the majority of deteriorations. This was followed by expiration (28 events), rapid response team (RRT) activations (22 events), and intubation events (19 events). Cardiac arrest was rare, with a single documented event.

In experiments, the lead time and sampling rate were analyzed. To characterize the timing of predictions relative to alerts, the lead times of predictions of the wearable-based RNN for each alert were evaluated against hard outcome. Here the lead time was defined as the first true positive preceding alerts and hard outcomes. To determine the effect of sampling rate on prediction performance, we varied the inter-sample timing of vital measurements and measured the effect on overall performance. Data were downsampled from the original inter-sample interval of one minute down to three hundred minutes. Performance was measured by ROC AUC and PR AUC at each inter-sample interval.

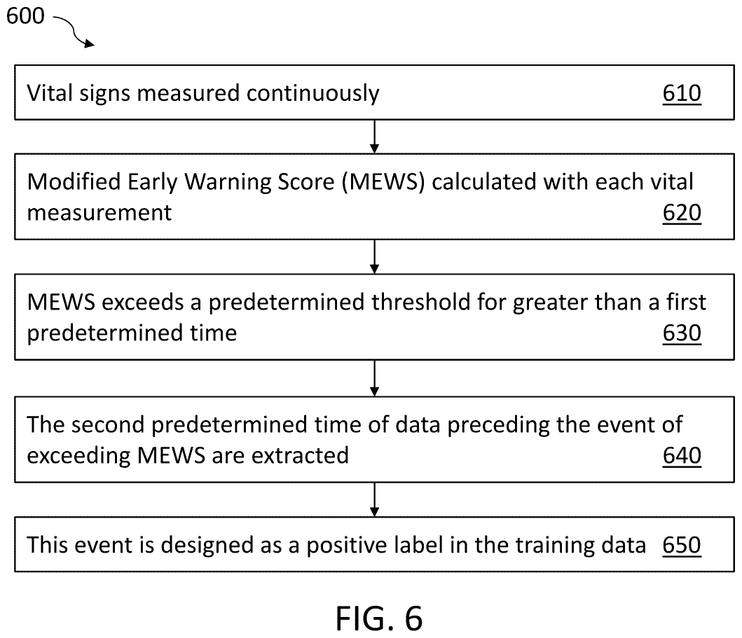

FIG. 6 is a flowchart illustrating a process 600 for health deterioration alert generation and training data acquisition according to embodiments of the present disclosure. The process 600 exemplarily includes blocks 610-650 as described herein below.

In block 610, vital signs of selected patients are measured continuously.

In block 620, a modified early warning score (MEWS) is calculated with each vital sign measurement.

In block 630, the MEWS is compared with a predetermined threshold, e.g., 6. If the MEWS exceeds the predetermined threshold for greater than a first predetermined time, e.g., 30 minutes, the process 600 proceeds to block 640.

In block 640, vital sign data measured during a second predetermined time preceding the event of exceeding MEWS are extracted.

In block 650, the event of exceeding MEWS is designated as a positive label in the training data.

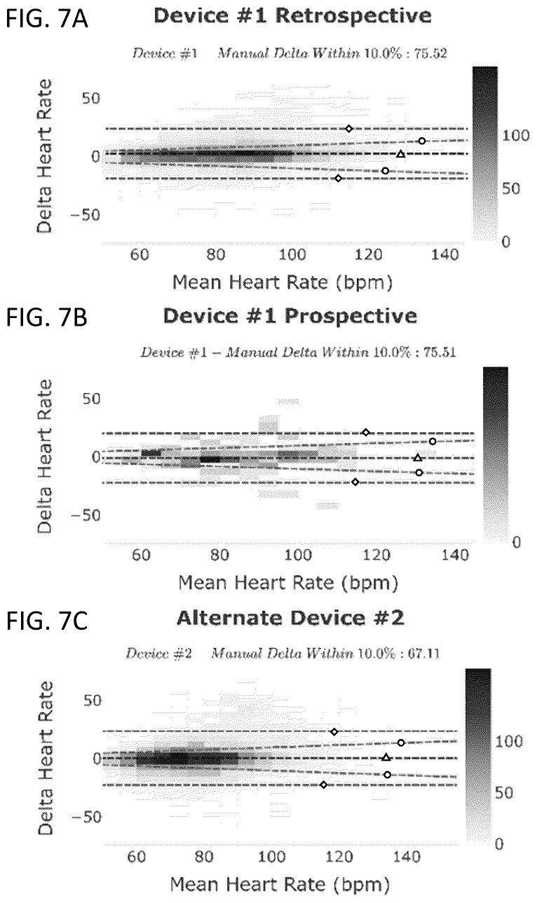

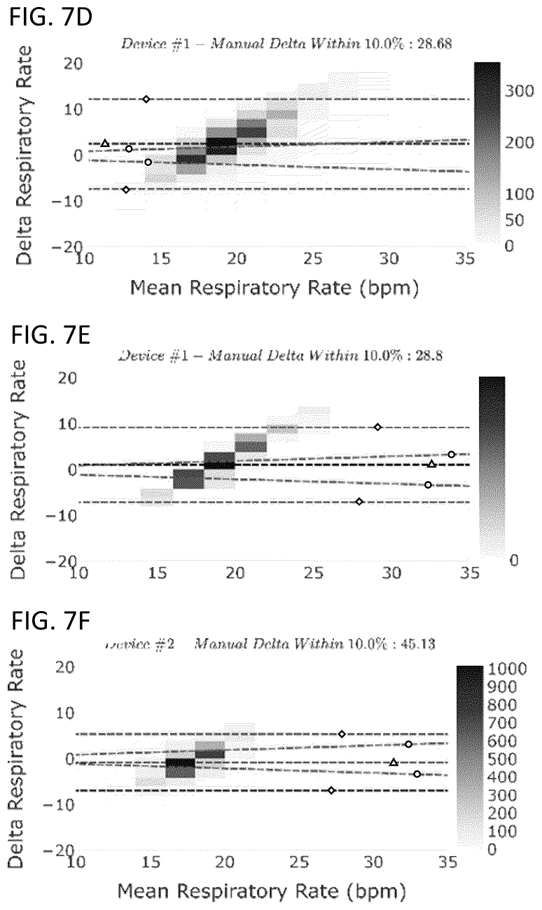

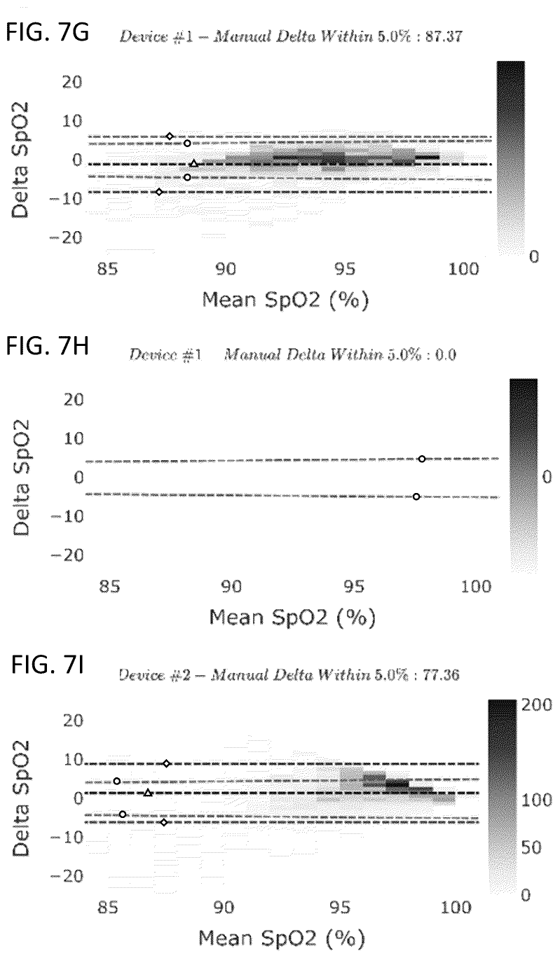

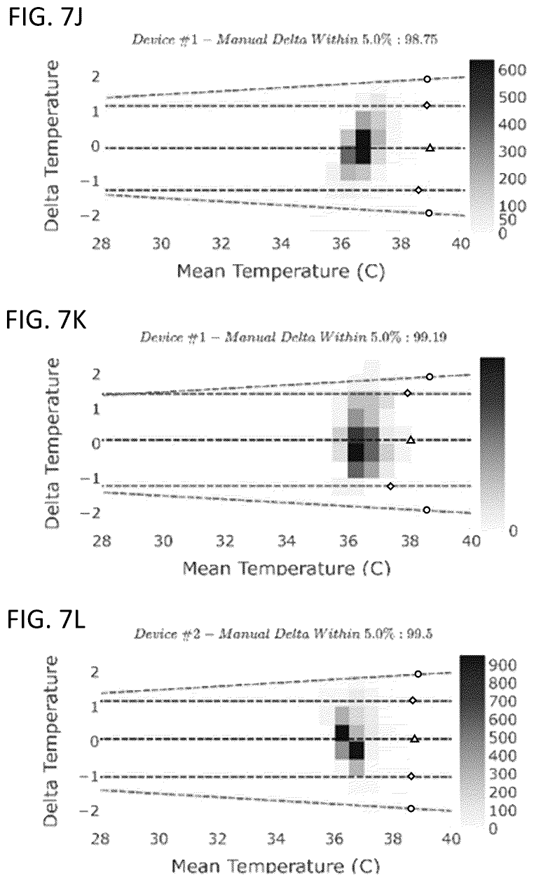

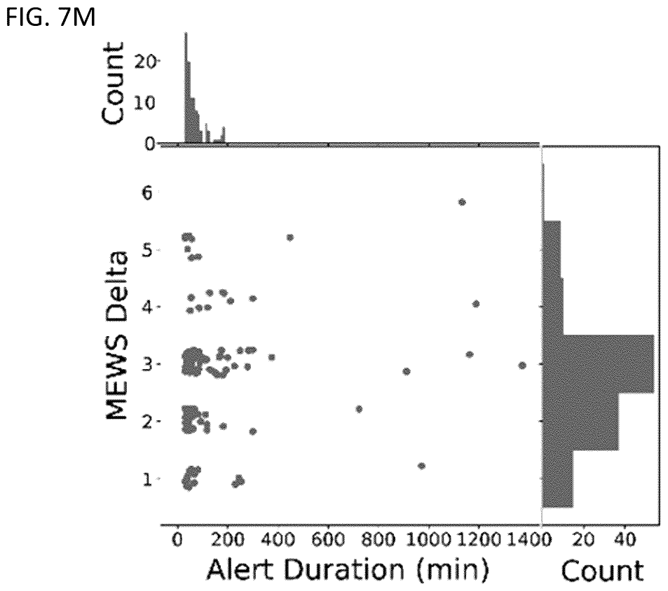

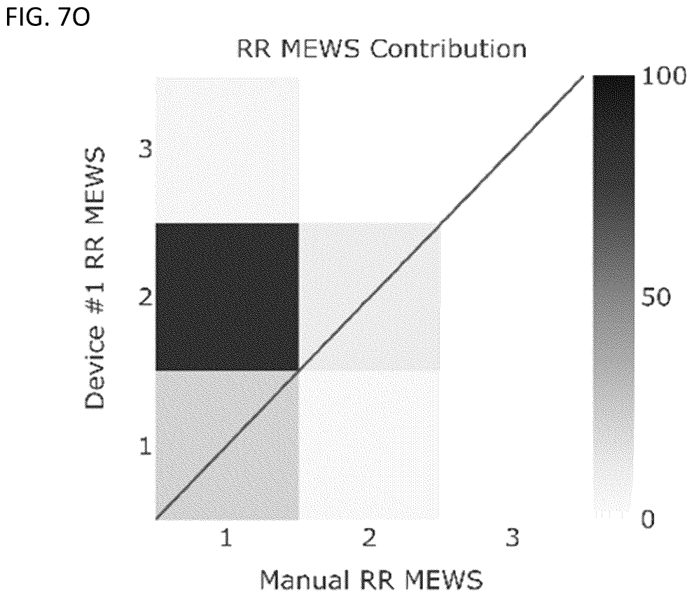

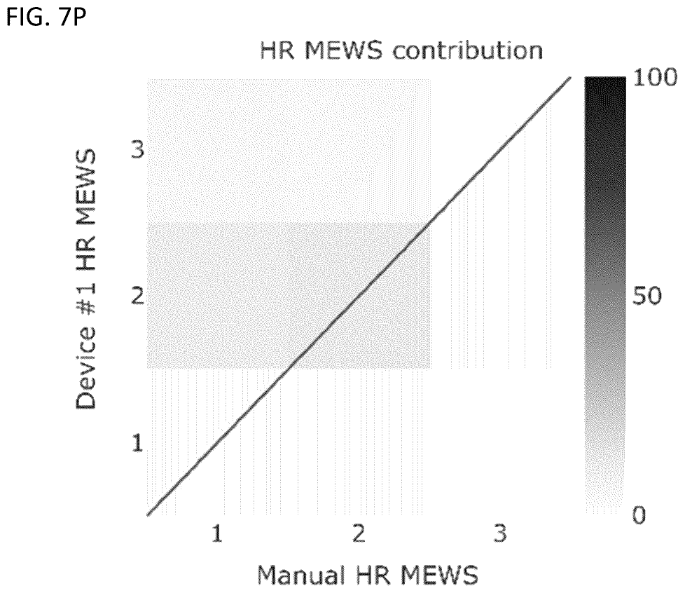

FIGS. 7A-7L are Bland-Altman plots illustrating wearable data evaluation according to embodiments of the present disclosure. Wearable (Wrbl) based Recurrent Neural Network (RNN), Logistic Regression (LogReg) and EHR based model performances illustrated by receiver operator characteristic (ROC) and precision-recall (PR) curves for twenty-four types of validation. Referring to FIG. 7G-7J, lead time analysis of wearable based RNN model illustrates how far in advance model can predict both clinical alerts and hard outcomes.

ROC and PR curves for predicting periods 30 mins or greater of continuously elevated MEWS (MEWS>6) 0.5-24 hours into the future. AUCs for each curve are listed in the figure legend. (FIG. 7A, 7B) Retrospective RNN and LR model performances in predicting an elevated MEWS using randomly shuffled Device #1 data, and concurrently collected EHR data, which was held out for testing. Performance of each model, for each dataset, across folds is plotted (lighter shade). Mean performances are plotted (darker shade) with one standard deviation of performances across folds also plotted (triangle). Chance performance of a binary classifier is plotted (dotted diamond line). (FIGS. 7C, 7D) RNN and LR Model performances in predicting an elevated MEWS using prospective data from the same device (Device #1 Prospective) and concurrently collected EHR data, which were both held out entirely for testing. (FIGS. 7E, 7F) RNN and LR Model performances in predicting an elevated MEWS using data from another device (Alternate Device #2), and concurrently collected EHR data, which were both held out entirely for testing. Lead times here are the timing of the first true positive preceding an alert. ‘ICU Trans’=Unplanned ICU Transfer, ‘Arrest’=Cardiac Arrest, ‘Vent’=Ventilator, ‘RRT’=Rapid response team call (FIG. 7G) Raster plot visualization of timing of all true positive predictions made by RNN model with wearable data preceding a clinical alert (Time 0). The symbols represent the various datasets—diamond raster: Device #1 Retrospective dataset, circle raster: Device #1 Prospective dataset, triangle raster: Alternate Device #2 dataset. (FIG. 7H) Probability density histogram of the lead times of the RNN model predictions for each alert. Shadings correspond to those in the raster plot. (FIG. 7I) Raster plot visualization of timing of all true positive predictions relative to hard outcomes (Time 0). (FIG. 7J) Probability density histogram of the lead times of the RNN model predictions for each hard outcome.

As shown in FIGS. 7A-7L, these Bland Altman plots provide comparisons between vital measurement from the CM devices and EHR vitals, for the retrospective, prospective, and alternate device datasets. Dotted diamond lines indicate the 95% confidence intervals of the deltas between wearable measurements and EHR measurements. Dotted triangle lines indicate the mean delta between wearable and EHR measurements. Circle dotted lines indicate a 10% deviation of the wearable measurement from the EHR measurement at that mean value. Sub-panel titles detail the percentage of vital measurements that fall within the 10% deviation line (circle dotted line). Bland Altman plots of (FIGS. 7A-6C) Device #1 retrospective HR, Device #1 Prospective HR, and Alternate Device #2 HR; (FIGS. 7D-6E) Device #1 Retrospective RR, Device #1 Prospective RR and Alternate Device #2 RR; (FIG. 7G-7I) Device #1 Retrospective SpO2, Device #1 Prospective SpO2 (Device #1 Prospective dataset did not have SpO2) and Alternate Device #2 SpO2. (FIGS. 7J-6L) Device #1 Retrospective temperature, Device #1 Prospective temperature, and Alternate Device #2 temperature.

Vital sign measurements from the wearable devices were evaluated by synchronizing and comparing with manually acquired vital signs logged in the EHR by constructing the Bland-Altman plots (FIGS. 7A-7L). The experiments used wearable vitals for modeling when 67% of measured values were within 10% error from the EHR values.

The Retrospective and Prospective Device #1 and Alternate Device #2 HR respectively had 75%, 75%, and 67% of measurements falling within the 10% deviation line, indicating sufficient HR concordance for use in modeling (FIGS. 7A, 7B, 7C). Retrospective and Prospective Device #1 and Alternate Device #2 RR respectively had 29%, 29%, and 45% of measurements falling within the 10% deviation line (FIGS. 7D, 7E, 7F). As EHR RR is often inaccurately measured, our inclusion criteria were not applied for this vital sign. Retrospective Device #1 and Alternate Device #2 SpO2 respectively had 87% and 77% of measurements falling within the 5% deviation line (FIGS. 7G, 7I), while Prospective Device #1 did not include SpO2 measurements (FIG. 7H). Retrospective and Prospective Device #1 and Alternate Device #2 temperature had 99% of all datasets' and devices' measurements fall within the 5% deviation line (FIGS. 7J, 7K, 7L). The threshold for acceptable agreement between the two measurements for both temperature and SpO2 readings was set at 5%, as ranges of normal values for these measurements are lower in clinical practice.

FIGS. 7M, 7O and 7P are plots illustrating clinical alert, vital contribution, and movement analysis according to embodiments of the present disclosure. These plots provide features of elevated MEWS score alerts, detected by CM device and missed by EHR, and contribution of CM vitals in their generation.

Two sets of wearable clinical alerts were generated post-hoc using vitals from Device #1 and EHR, respectively. Differences in the resulting alert onsets and magnitudes were compared between these two sets. (FIG. 7M) Scatterplot and marginal distributions of the duration of wearable clinical alerts (MEWS>6) that were detected by the wearable but missed by the EHR, along with the difference in magnitude of the MEWS score, between wearable vs EHR derived MEWS, at alert onset. (FIG. 7O, 7P) Heatmaps of MEWS contribution of RR. The wearable MEWS score was plotted on the y-axis and the EHR MEWS score on the x-axis. The diagonal circle line represents equal scores given to both the wearable- and EHR-based vitals. Off diagonal values indicate that one modality was given a higher score than the others. The experiments are focused on respiratory rate and heart rate measurements since both the temperature and SpO2 measurements were congruent between EHR and CM values. (FIG. 7O) and HR (FIG. 7P) from the wearable device (y-axis) against the EHR (x-axis, labelled ‘Manual’).

Two sets of clinical alerts were generated post hoc using vitals from Device #1 and the EHR, respectively, with differences in the resulting alert volumes and magnitudes compared. The alert algorithm detected 140 clinical alerts, defined as a MEWS greater than 6 for 30 minutes or longer. The marginal histogram plot (FIG. 7M) shows the number of elevated wearable-MEWS score events (not appearing in the EHR) plotted by delta in alert magnitude (wearable-MEWS-EHR-MEWS [x-axis]) against alert duration (y-axis). The CM devices detected 126 (or 9×) more alerts than manual monitoring (FIG. 7M). Of all alerts, 90% were detected by Device #1 alone, while fewer than 3% were only detected in the EHR. For 7% of alerts that were detected by both, the wearable-based alerts preceded the EHR alerts by an average of 105 minutes.

The incongruity in alerts between wearable- and EHR-derived MEWS alerts was due to RR (FIG. 7O) and HR (FIG. 7P) measurements, contributing to higher MEWS scores and thus more alerts. In plotting the respective MEWS contributions of the vital signs from each modality against each other, RR and HR respectively contributed 30× and 10× as much to the MEWS scores as compared with other vital signs. For the 126 (i.e., 90%) of alerts that were missed by the EHR, accurate reporting of RR accounted for 89 (i.e., 71%) of these alerts, while elevated continuous HR measures accounted for 20 (i.e., 16%).

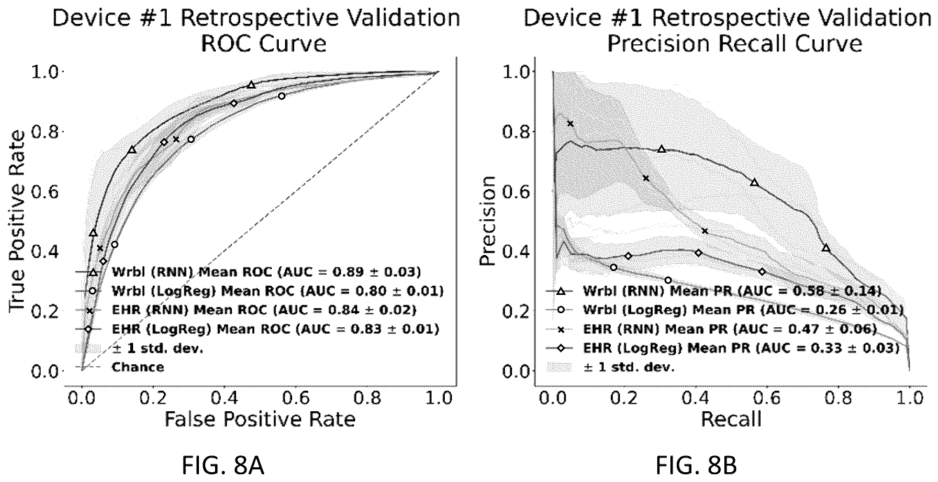

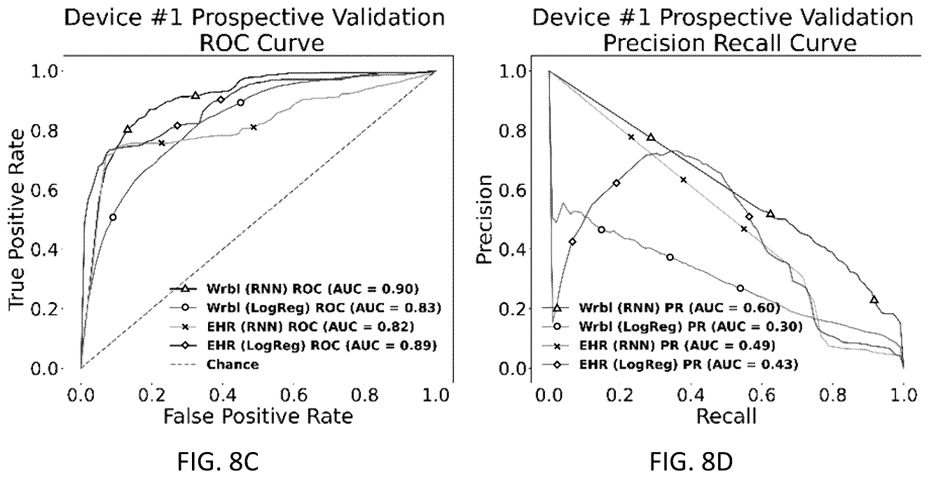

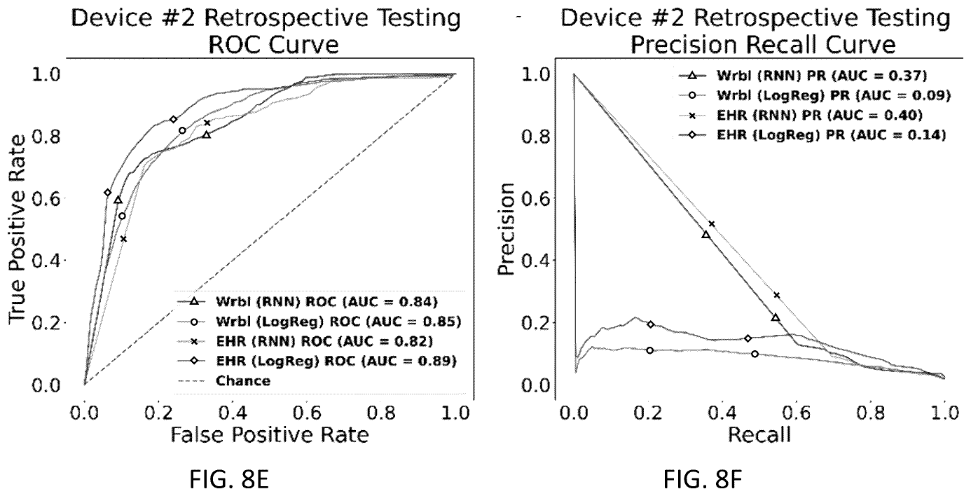

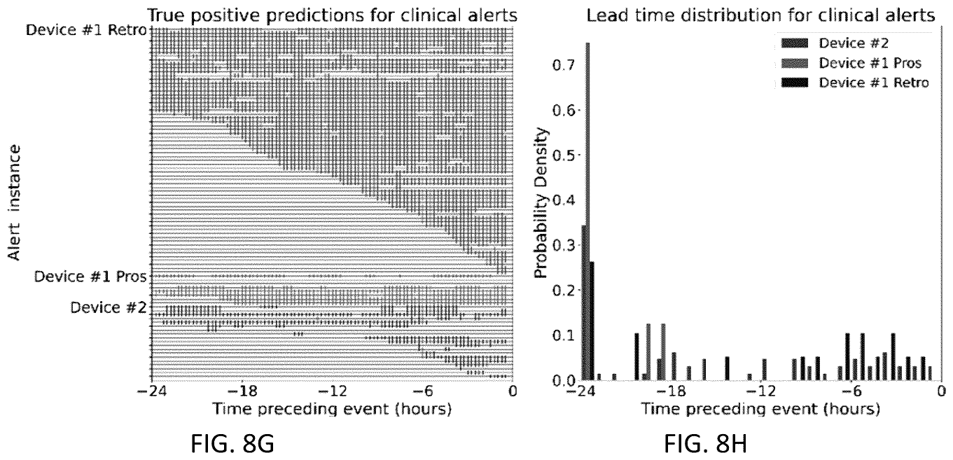

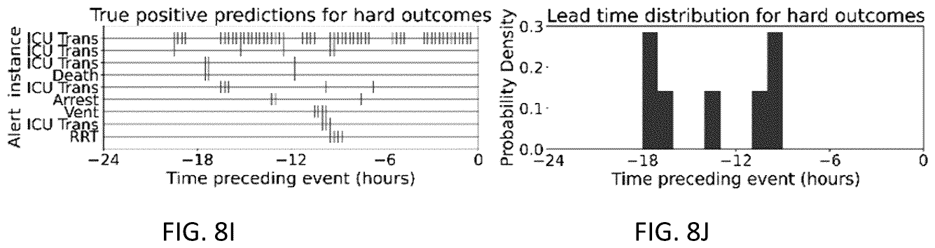

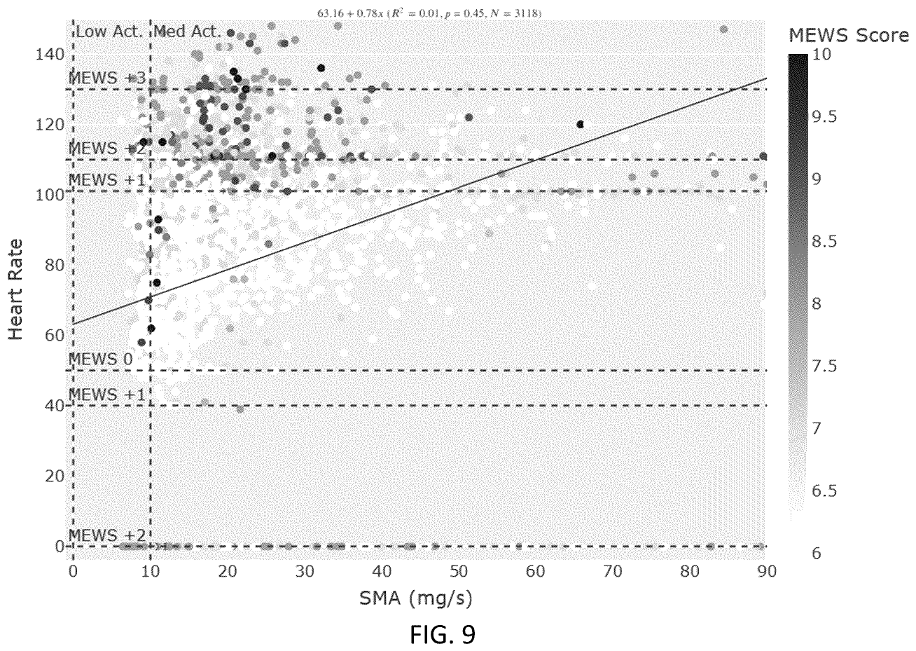

Since HR and RR can change significantly with patient movement, we sought to evaluate whether movement-induced elevation of these two vitals might be contributing to elevated MEWS values (i.e., alerts) despite clinical and physiologic patient stability. We examined the values of the vitals (i.e., HR, RR) against the average SMA values over the duration of the detected alert and associated MEWS value. The lack of a correlation between increasing movement (i.e., SMA values) and increasing vital sign measurements (i.e., HR−R²=0.01, RR−R²=0.02) or MEWS values indicated that the alerts were generated independently of movement (referring to FIGS. 8 and 9).

In experiments, the clinical alert modeling performance and validation were evaluated. The experiments included training LSTM deep learning model using various demographic and continuously monitored vital sign features to predict in-hospital deterioration with a 24-hour time horizon using a 5-hour sequence. A logistic regression (LR) based model was also trained for comparison, and all models were compared to a dataset using EHR data alone without continuous device data. The predictive performance of these models was evaluated retrospectively (i.e., Device #1 held out data), prospectively (i.e., Device #1 separate hospital), and externally (i.e., Device #2 alternate device). Setting an operating point to achieve a positive predictive value of 0.2, we also examined the confusion matrix for each of the three validation steps (referring to TABLE 2).

FIGS. 8A and 8B are plots illustrating retrospective validation on Device #1 data according to embodiments of the present disclosure. Using data held out for testing from Device #1, experiments have been conducted to validate the RNN-based model to predict the occurrence of CM-based clinical alerts. The RNN model performance on this retrospective dataset (triangle trace) had mean±/−SD ROC AUC 0.89+/−0.03 and PR AUC 0.58+/−0.14 as compared to the LR model (mean ROC AUC 0.80+/−0.01 and PR AUC 0.26+/−0.01, circle trace-FIGS. 8A and 8B and TABLE 2). The RNN-based model had worse performance when using EHR data alone (x-shaped trace-FIGS. 8A and 8B) versus device data with mean ROC AUC 0.84+/−0.02 and PR AUC of 0.47+/−0.06 compared to the LR model with EHR data (diamond trace-FIGS. 8A and 8B) with mean ROC AUC 0.83+/−0.01 and PR AUC of 0.33+/−0.03).

FIGS. 8C and 8D are plots illustrating prospective validation on Device #1 data according to embodiments of the present disclosure. Using the prospective dataset held out entirely for testing from Device #1 (separate hospital), the model performance on this retrospective dataset (triangle trace-FIGS. 8C and 8D) had ROC AUC 0.9 and PR AUC 0.6, with LR model showing worse performance (ROC AUC 0.83 and PR AUC 0.3, circle trace-FIGS. 8C and 8D and TABLE 2). The RNN-based model using EHR data (circle trace-FIGS. 8C and 8D) alone showed a degradation in performance with ROC AUC 0.82 and PR AUC 0.49 and LR model with EHR data (diamond trace-FIGS. 8C and 8D) having ROC AUC 0.89 and PR AUC 0.43.

FIGS. 8E and 8F are plots illustrating alternate device testing on Device #2 according to embodiments of the present disclosure. All data from Alternate Device #2 was held out for testing and used for external validation of the RNN-based model. The model performance showed (triangle trace-FIGS. 8E and 8F) ROC AUC 0.84 and PR AUC 0.37 (LR model ROC AUC 0.85 and PR AUC 0.09, circle trace-FIGS. 8E and 8F and TABLE 2). Using EHR data to predict alerts, the RNN-based model (circle trace, FIGS. 8E and 8F) had ROC AUC 0.82 and PR AUC 0.4 (LR with EHR model; diamond trace-FIGS. 8E and 8F, ROC AUC 0.89 and PR AUC 0.14).

FIGS. 8G-7J are plots illustrating lead time and sampling rate analysis according to embodiments of the present disclosure. To characterize the timing of predictions relative to alerts, experiments were conducted to examine lead times of predictions of the RNN model for each alert and hard outcome. Lead times are the first true positive preceding actual alerts and hard outcomes. The experiments visualized the true positives on a raster plot that showed all RNN-based alerts 24 hours preceding a clinical alert (FIG. 8G) along with the distribution of lead times (FIG. 8H). For the three stages of validation-Device #1 retrospective, Device #1 prospective, Device #2 alternate—the median and interquartile range (IRQ) of lead time was 17.12 hours [6.81−24.0], 24.0 hours [22.88−24.0] and 8.25 hours [5.25−21.75], respectively. For this analysis, the experiments included limiting the scope of true positives to 24 hours, though several alerts for each validation had lead times greater than 24 hours. The percentage of alerts whose lead time was greater than 24 hours for Device #1 retrospective, Device #1 prospective, Device #2 alternate was 14%, 75%, and 21%, respectively.

The experiments included repeating this analysis for hard outcome predictions with all true positives preceding an outcome being visualized (FIG. 8I). The timing of these first true positives is plotted in a probability density histogram plot with median (IQR) lead time of 16.5 [10.5−17.5] (FIG. 8J).

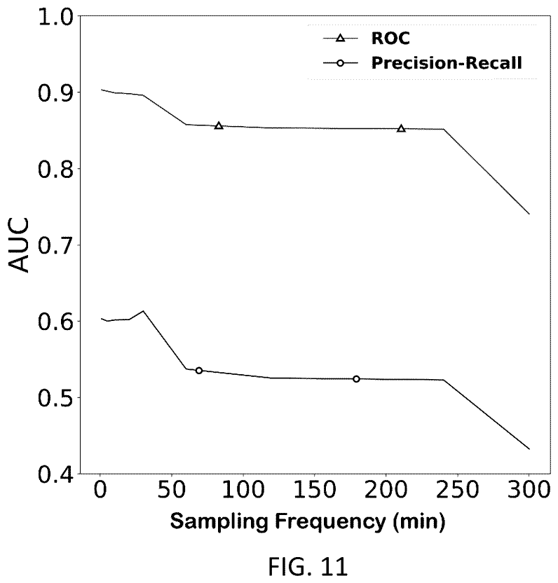

To determine the effect of sampling rate on prediction performance, experiments were conducted to vary the inter-sample timing of vital measurements from one to three hundred minutes and measured the effect on ROC AUC and PR AUC at each inter-sample interval (FIG. 11). Going from one to three hundred minutes, the ROC AUC dropped 16.2% and the PR AUC dropped 17.0%. The ROC and PR AUC maximum occurs at inter-sample intervals of one and thirty minutes, respectively.

TABLE 4
cutoff values for various classes of movement derived
from Device #1's signal magnitude area (SMA) signal
which is the average output of a 3-axis accelerometer.

| | SMA values (mg/s) | | |
|---|---|---|---|
| Percentiles | Stationary | ADL | Strenuous |
| 25 | 8 | 10 | 94 |
| 50 | 8 | 26 | 262 |
| 75 | 10 | 118 | 456 |

TABLE 5
Modified Early Warning Score (MEWS) calculation, based upon vital sign measurements.
Each item is given a score, and the final score is tallied (range 0-15), with
higher values indicating greater risk of clinical decompensation.

| | 3 | 2 | 1 | 0 | 1 | 2 | 3 |
|---|---|---|---|---|---|---|---|
| Systolic blood | <70 | 70-80 | 81-100 | 101-199 | | ≥200 | |
| pressure | | | | | | | |
| (mmHg) | | | | | | | |
| Heart rate | | <40 | 41-50 | 51-100 | 101-110 | 110-129 | ≥130 |
| (bpm) | | | | | | | |
| Respiratory | | <9 | | 9-14 | 15-20 | 21-29 | ≥30 |
| rate (bpm) | | | | | | | |
| Temperature | | <35 | | 35-38.4 | | ≥38.5 | |
| Neurological | | | | Alert | React | React | Unresponsive |
| status (AVPU) | | | | | voice | Pain | |
| BMI (kg/m²) | | | <18.5 | | 25.1-34.9 | ≥35 | |
| Age (year) | | | | | 65-74 | 75-84 | ≥85 |

TABLE 6
health system protocol as defined by the Modified Early Warning Score
(MEWS) value, incorporated into the electronic health record.

| | Health system protocol recommended action |
|---|---|
| MEWS 7 | Increase vital sign frequency to every 2 hours |
| MEW 8 | Increase vital sign frequency to every 2 hours + |
| | licensed independent provider evaluation |
| MEWS 9 | Increase vital sign frequency to every 2 hours + |
| | licensed independent provider evaluation + |
| | consider rapid response team evaluation |
| MEWS 10+ | Increase vital sign frequency to every 2 hours + |
| | licensed independent provider evaluation + |
| | consider rapid response team evaluation + |
| | change in level of service |

To estimate the sample size necessary for validating the wearable data in present disclosure, Cohen's d statistic was calculated using the following formula:

$$d = \frac{\overline{X 1} - \overline{X 2}}{SD}$$

Where X̅1̅ is the mean of the distribution of vital values measured from the wearable device and X̅2̅ is the mean of the distribution of vital values measured in the EHR. SD represents the expected standard deviation of the grouped data. Using this formula, we set out to test whether the difference in the means of the wearable and EHR vital values exceeded 10%, in other words, whether there was a 10% error or greater. To give a concrete example, assuming an expected average heart rate of 75 bpm, a 10% difference in the means of the heart rates, from wearable and EHR sources, would translate to a difference of the means of at least 7.5 bpm. The expected aggregate standard deviation of the heart rate from both sources is approximately 10 bpm. Entering these values into the equation above results in a Cohen's d of 0.75. For this study, we chose a power level of 95% with alpha=0.01. Using these parameters and the following sample size lookup table:

| | $\frac{a 2 = \text{.01} ( a 1 = 0.005 )}{d}$ | | | | | | | | | | |
|---|---|---|---|---|---|---|---|---|---|---|---|
| Power | .10 | .20 | .30 | .40 | .50 | .60 | .70 | .80 | 1.00 | 1.20 | 1.40 |
| .25 | 725 | 183 | 82 | 47 | 31 | 22 | 17 | 13 | 9 | 7 | 6 |
| .50 | 1329 | 333 | 149 | 85 | 55 | 39 | 29 | 22 | 15 | 11 | 9 |
| .60 | 1603 | 402 | 180 | 102 | 66 | 46 | 34 | 27 | 18 | 13 | 10 |
| 2/3 | 1810 | 454 | 203 | 115 | 74 | 52 | 39 | 30 | 20 | 14 | 11 |
| .70 | 1924 | 482 | 215 | 122 | 79 | 55 | 41 | 32 | 21 | 15 | 12 |
| .75 | 2108 | 528 | 236 | 134 | 86 | 60 | 45 | 35 | 23 | 17 | 13 |
| .80 | 2338 | 586 | 259 | 148 | 95 | 67 | 49 | 38 | 25 | 18 | 14 |
| .85 | 2611 | 654 | 292 | 165 | 106 | 74 | 55 | 43 | 28 | 20 | 15 |
| .90 | 2978 | 746 | 332 | 188 | 120 | 84 | 62 | 48 | 31 | 22 | 17 |
| .95 | 3564 | 892 | 398 | 224 | 144 | 101 | 74 | 57 | 37 | 26 | 20 |
| .99 | 4808 | 1203 | 536 | 302 | 194 | 136 | 100 | 77 | 50 | 35 | 26 |

Resulted in an estimated required sample size between 57 and 74. Since each of our participants had many measurements, our study far exceeded the required sample size to detect the error rate in vital sign measurement between the wearable device and EHR.

In addition to estimating the sample size for validating the wearable data, we also estimated the amount of data necessary for neural network convergence. The choice of cohort size was made based on other clinical studies with similar architectures, 59, 60 our prior studies, 61 as well as studies on VC dimensions of neural networks. With this context in mind, we observed that LSTM RNNs typically converge on sample sizes with over 600 patients. Recruiting 888 patients was based on this consideration and the availability of patches during this study.

TABLE 7
Missing data from CM devices from
preliminary data from 888 patients.

| Missingness (%) | VitalPatch | Biobeat |
|---|---|---|
| Respiratory Rate | 3.72 | 8.3 |
| Heart Rate | 3.72 | 0 |
| Body Temp. | 0 | 3.9 |
| SpO2 | 14.95 | 23.02 |
| Activity (SMA) | 0 | 0 |

FIG. 9 shows an exemplary relationship between signal magnitude area (SMA) and heart rate according to embodiments of the present disclosure. To evaluate whether movement (SMA) may be contributing to vital sign derangement and thus elevated MEWS values, we examined the values of HR against the average SMA values over the duration of the detected alert and associated MEWS value. The alerts and their corresponding average SMA values were grouped into two classes (vertical dotted lines): “stationary” or Low Activity (Low Act.) and “activities of daily living” or Medium Activity (Med. Act.), based on manufacturer determined SMA cutoff values (TABLE 4). The heart rate was then also grouped into six classes depending on its point contribution (TABLE 5) to the composite MEWS score (horizontal dotted lines). Each SMA-HR pair was then shaded by the average MEWS score over the duration that the MEWS score was elevated for that alert. The linear fit equation, correlation coefficient and significance sit at the top of the figure.

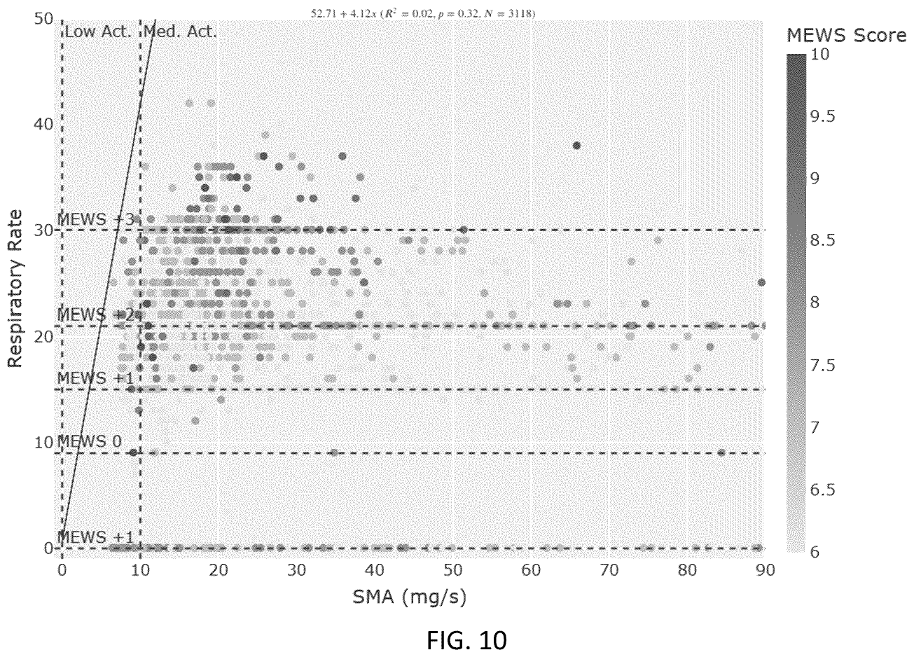

FIG. 10 shows an exemplary relationship between movement (SMA) and respiratory rate (RR) according to embodiments of the present disclosure. To evaluate whether movement (SMA) may be contributing to vital sign derangement and thus elevated MEWS values, we examined the values of RR against the average SMA values over the duration of the detected alert and associated MEWS value. The alerts and their corresponding average SMA values were grouped into two classes (vertical dotted lines): “stationary” or Low Activity (Low Act.) and “activities of daily living” or Medium Activity (Med. Act.), based on manufacturer determined SMA cutoff values (TABLE 4). The RR was then also grouped into five classes depending on its point contribution (TABLE 5) to the composite MEWS score (horizontal dotted lines). Each SMA-RR pair was then shaded by the average MEWS score over the duration that the MEWS score was elevated for that alert. The linear fit equation, correlation coefficient and significance sit at the top of the figure.

FIG. 11 shows an exemplary relationship between inter-sampling interval and performance of the wearable based RNN deterioration prediction algorithm according to embodiments of the present disclosure. ROC AUC (triangle trace) and PR AUC (circle trace) metrics show the effect of inter-sample interval on prediction performance as the inter-sample interval of vital measurements is extended out from 1-minute to 300 minutes.

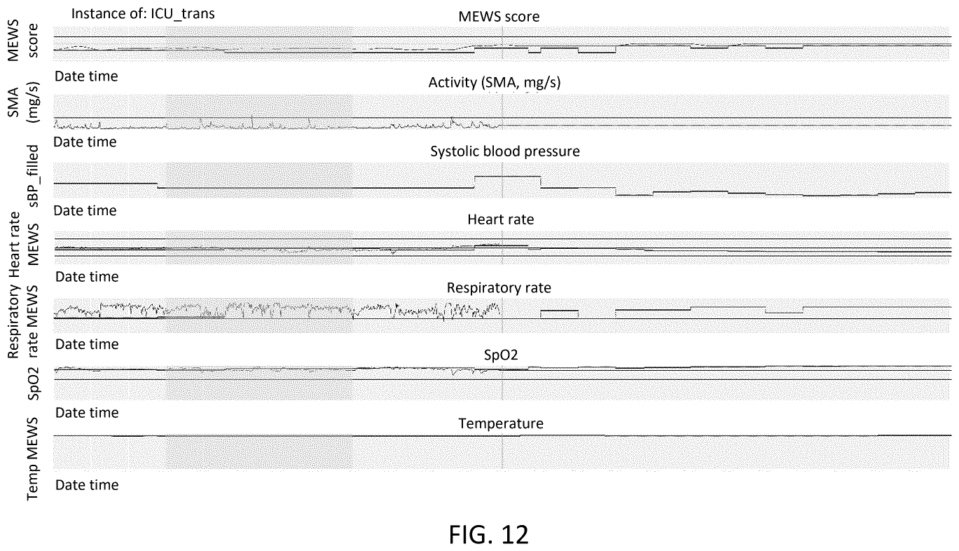

FIG. 12 shows sample patient vital signs preceding an ICU transfer even according to embodiments of the present disclosure. Displayed below are the patient's vital signs prior to the ICU transfer event: manually measured vital values input by a nurse into the EHR, the most recently calculated MEWS score based on all of the manually measured vitals in the EHR, the most recently measured wearable value for that particular vital, and the most recently calculated MEWS score based on the continuously measured vital signals from the wearable device. The black dotted line in the top plot represents the MEWS 6 threshold that was used for identifying clinical alerts. The vertical line indicates the time of the ICU transfer event. The shaded area represents an example of a 5-hour sequence of data that is extracted and passed into a model to try to predict the ICU transfer event, in this case with a three-hour lead time.

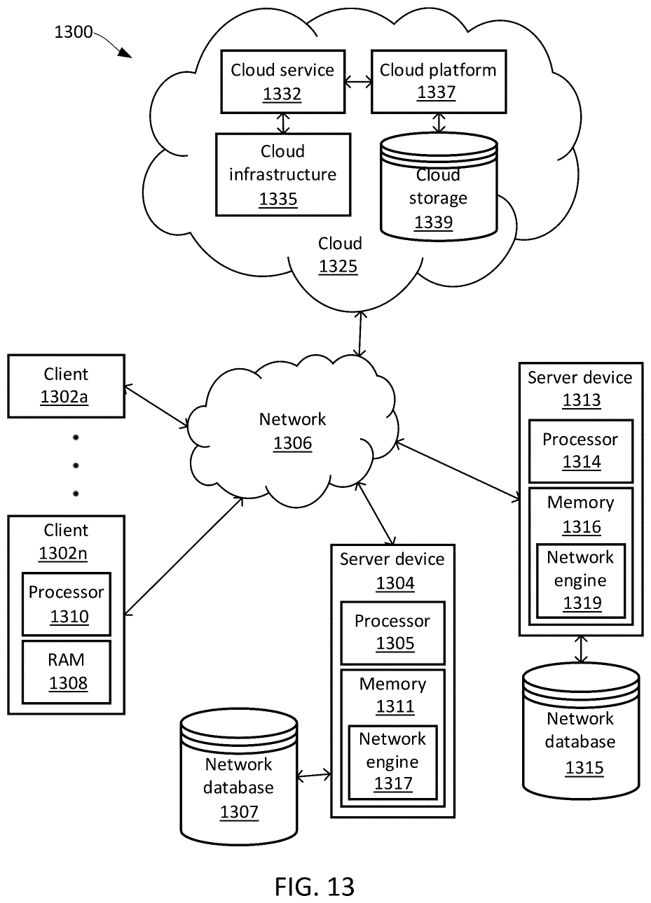

FIG. 13 is a block diagram illustrating an exemplary computer-based system/platform 1300 in accordance with one or more embodiments of the present disclosure. However, not all of these components may be required to practice one or more embodiments, and variations in the arrangement and type of the components may be made without departing from the spirit or scope of various embodiments of the present disclosure. In at least some embodiments, client computing devices 1302a through 1302n shown each at least includes a computer-readable medium, such as a random-access memory (RAM) 1308 coupled to a processor 1310 or FLASH memory (not shown) associated thereto. In at least some embodiments, processor 1310 may execute computer-executable program instructions stored in memory 1308. In at least some embodiments, processor 1310 may include a microprocessor, an ASIC, and/or a state machine. In at least some embodiments, processor 1310 may include, or may be in communication with, media, for example computer-readable media, which stores instructions that, when executed by processor 1310, may cause processor 1310 to perform one or more steps described herein. In at least some embodiments, examples of computer-readable media may include, but are not limited to, an electronic, optical, magnetic, or other storage or transmission device capable of providing a processor, such as the processor 1310 of client 1302a, with computer-readable instructions. In at least some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In at least some embodiments, the instructions may comprise code from any computer-programming language, including, for example, C, C++, Visual Basic, Java, Python, Perl, JavaScript, and etc.

In at least some embodiments, client computing devices 1302a through 1302n may also comprise a number of external or internal devices such as a mouse, a CD-ROM, DVD, a physical or virtual keyboard, a display, a speaker, or other input or output devices. In at least some embodiments, examples of client computing devices 1302a through 1302n (e.g., clients) may be any type of processor-based platforms that are connected to a network 1306 such as, without limitation, personal computers, digital assistants, personal digital assistants, smart phones, pagers, digital tablets, laptop computers, Internet appliances, and other processor-based devices. In at least some embodiments, client computing devices 1302a through 1302n may be specifically programmed with one or more application programs in accordance with one or more principles/methodologies detailed herein. In at least some embodiments, client computing devices 1302a through 1302n may operate on any operating system capable of supporting a browser or browser-enabled application, such as Microsoft™, Windows™, and/or Linux. In at least some embodiments, client computing devices 1302a through 1302n shown may include, for example, personal computers executing a browser application program such as Microsoft Corporation's Internet Explorer™, Apple Computer, Inc.'s Safari™, Mozilla Firefox, and/or Opera. In at least some embodiments, through the client computing client devices 1302a through 1302n, users may communicate over exemplary network 1306 with each other and/or with other systems and/or devices coupled to network 1306.

As shown in FIG. 13, exemplary server devices 1304 and 1313 may also be coupled to network 506. Exemplary server device 1304 may include a processor 1305 coupled to a memory 1311 that may store a network engine 1317. Exemplary server device 1313 may include a processor 1314 coupled to a memory 1316 that may store a network engine 1319. In at least some embodiments, one or more client computing devices 1302a through 1302n may be mobile clients.

As shown in FIG. 13, exemplary server device 1304 may be coupled to an exemplary network database 1307; and exemplary server device 1313 may be coupled to an exemplary network database 1315. In at least some embodiments, exemplary databases 1307 and 1315 may be any type of database, including a database managed by a database management system (DBMS). In at least some embodiments, an exemplary DBMS-managed database may be specifically programmed as an engine that controls organization, storage, management, and/or retrieval of data in the respective database. In at least some embodiments, the exemplary DBMS-managed database may be specifically programmed to provide the ability to query, backup and replicate, enforce rules, provide security, compute, perform change and access logging, and/or automate optimization. In at least some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In at least some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In at least some embodiments, the exemplary DBMS-managed database may be specifically programmed to include metadata about the data that is stored.

As shown in FIG. 13, network 1306 may be coupled to a cloud computing/architecture(s) 1325. Cloud computing/architecture(s) 1325 may include a cloud service 1332 coupled to a cloud infrastructure 1335 and a cloud platform 1337, where the cloud platform 1337 may be coupled to a cloud storage 1339.

The computing system can also employ any number of software, firmware, and/or hardware configurations. For example, one or more of the example embodiments disclosed herein can be encoded as a computer program (also referred to as computer software, software applications, computer-readable instructions, and/or computer control logic) on a computer-readable medium.

The term “computer-readable medium,” as used herein, can generally refer to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In at least some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment may be implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In at least some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

In at least some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21).NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In at least some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In at least some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

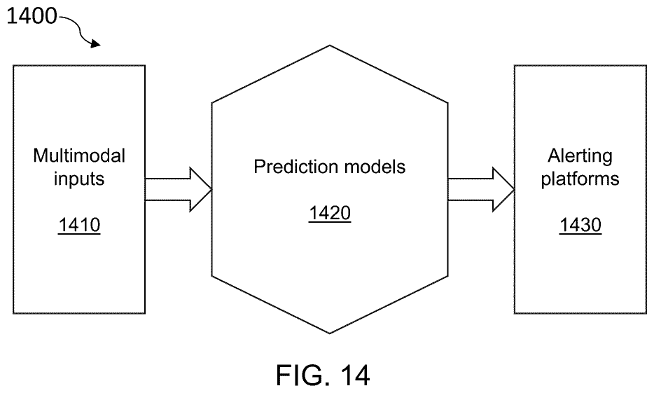

FIG. 14 is a block diagram illustrating a system 1400 for early detection of patient health deterioration according to embodiments of the present disclosure. The system 1440 exemplarily includes multimodal inputs 1410, prediction models 1420 and alerting platforms 1430. In at least some embodiments, the multimodal inputs 1410 include real-time data from both wearable devices and EHRs. The wearable devices examplerily include a Vital Connect vitalpatch, a Biobeat chest monitor, a Masimo radius vital sign monitor (VSM), a biointellisense boibutton, a Neteara 130H-plus, a Sibel health ANNE chest sensor, and an Isansys Lifetouch device, etc. The wearable devices continuously monitors the vital signs. The EHRs contain periodically measured vital signs which are measured manually with more precise equipment. In at least some embodiments, the multimodal inputs are provided to prediction models 1420 in real-time.

As shown in FIG. 14, the multimodal inputs 1410 are provided to prediction models which are, for example, trained machine learning models that can make inferences based on the input data. In at least some embodiments, new, unseen data is processed in the same way as the training data. This ensures consistency in how the model interprets the data.

As shown in FIG. 14, the alerting platform 1430 receives the inferences generated by the prediction model and generate early detection of patient heath deteriorations based at least on the inferences and a predetermined set of rules. In at least some embodiments, the alerting platform 1430 is integrated with other systems such as an Epic Systems™ from Verona, Wisconsin, Teams™ from Microsoft in Redmond WA, IOS™ from Apple in Cupertino CA, and/or Slack™ from Salesforce in San Francisco CA. In at least some embodiments, the alerting platform 1430 offers application programming interfaces (APIs) for third-party application software development.

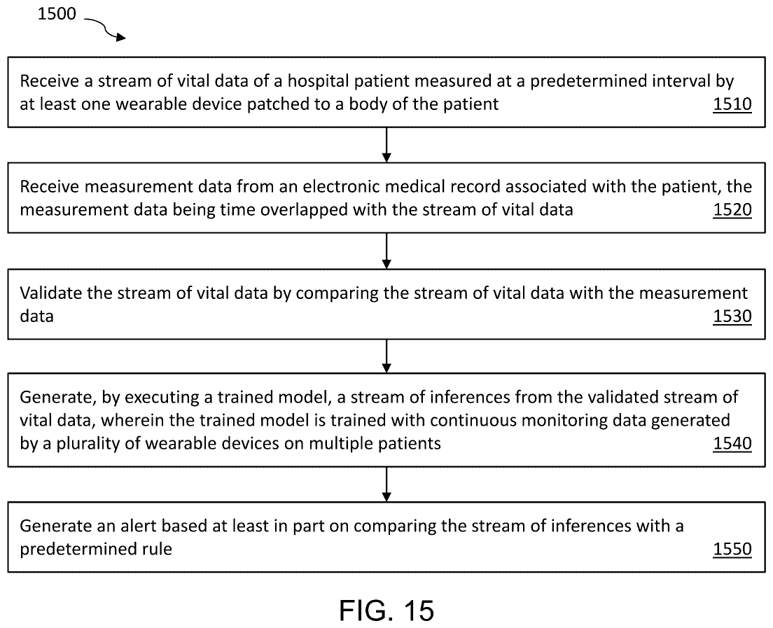

FIG. 15 is a flowchart illustrating a process 1500 for generating health deterioration alert according to embodiments of the present disclosure. The process 1500 exemplarily includes blocks 1510-1550 as described herein below.

In block 1510, the process 1500 includes receiving a stream of vital data of a hospital patient measured at a predetermined interval by at least one wearable device patched to a body of the patient.

In block 1520, the process 1500 includes receiving measurement data from an electronic medical record associated with the patient, the measurement data being time overlapped with the stream of vital data, i.e., the stream of vital data and the measurement data are measured during proximately the same time period.

In block 1530, the process 1500 includes validating the stream of vital data by comparing the stream of vital data with the measurement data.

In block 1540, the process 1500 includes generating, by executing a trained model, a stream of inferences from the validated stream of vital data, wherein the trained model is trained with continuous monitoring data generated by a plurality of wearable devices on multiple patients.

In block 1550, the process 1500 includes generating an alert based at least in part on comparing the stream of inferences with a predetermined rule.

The aforementioned examples are, of course, illustrative, and not restrictive.

As used herein, the term “user” shall have a meaning of at least one user. In at least some embodiments, the terms “user”, “subscriber” “consumer” or “customer” should be understood to refer to a user of an application or applications for implementing the functions of the CVCP as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative, and not restrictive.

In at least some embodiments, the exemplary inventive computer-based systems, the exemplary inventive computer-based devices, and/or the exemplary inventive computer-based components of the present disclosure may be configured to utilize one or more exemplary AI/machine learning techniques chosen from, but not limited to, decision trees, boosting, support-vector machines, neural networks, nearest neighbor algorithms, Naïve Bayes, bagging, random forests, and the like. In at least some embodiments and, optionally, in combination of any embodiment described above or below, an exemplary neural network technique may be one of, without limitation, feedforward neural network, radial basis function network, recurrent neural network, convolutional network (e.g., U-net) or other suitable network. In at least some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In at least some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In at least some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node may be activated. In at least some embodiments and, optionally, in combination of any embodiment described above or below, the exemplary aggregation function may be a mathematical function that combines (e.g., sum, product, etc.) input signals to the node. In at least some embodiments and, optionally, in combination of any embodiment described above or below, an output of the exemplary aggregation function may be used as input to the exemplary activation function. In at least some embodiments and, optionally, in combination of any embodiment described above or below, the bias may be a constant value or function that may be used by the aggregation function and/or the activation function to make the node more or less likely to be activated.

The aforementioned examples are, of course, illustrative, and not restrictive.

At least some aspects of the present disclosure will now be described with reference to the following numbered clauses.

Clause 1. A system configured for early-detection of an event of health condition deterioration of a subject, including: at least one first wearable device patched to a body of a person for measuring at least one vital sign thereof; a controller configured to schedule the at least one first wearable device to continuously measure the at least one vital sign of the person at a first predetermined interval to generate a first stream of vital data; a signal processing engine configured to receive the first stream of vital data from the at least one first wearable device and measurement data from an electronic medical record associated with the person, the measurement data being time overlapped with the first stream of vital data, the signal processing engine configured to validate the first stream of vital data by comparing the first stream of vital data with the measurement data; a trained model configured to receive the validated first stream of vital data to generate a stream of inferences, wherein the trained model is trained with continuous monitoring data generated by a plurality of wearable devices on multiple persons; and an alert generator configured to generate an alert signal, to a monitoring system associated with the person, based at least in part on comparing the stream of inferences to a predetermined rule, the predetermined rule including a threshold of a modified early warning score (MEWS) and a duration of the stream of inferences being above the threshold, or a discrete timing of a set of hard clinical outcomes including but not limited to: cardiac arrests, intubations, rapid response team calls, unplanned ICU transfers and expirations.

Clause 2. The system of clause 1, wherein the measurement data are generated by episodically measuring vital signs of the person using standard hospital equipment.

Clause 3. The system of clause 2, wherein the measurement data is selected from systolic blood pressure (SBP), heart rate (HR), respiratory rate (RR), temperature (Temp), oxygen saturation (SpO2), level of consciousness (AVPU) and any combination thereof.

Clause 4. The system of clause 1, wherein the signal processing engine is further configured to reject at least one artifact from the first stream of vital data for validating the first stream of vital data.

Clause 5. The system of clause 4, wherein the at least one artifact includes data assumed of physiologically implausible values.

Clause 6. The system of clause 1, further including at least one second wearable device controlled by the controller to continuously measure the at least one vital sign of the person at a second predetermined interval to generate a second stream of vital data.

Clause 7. The system of clause 6, wherein the signal processing engine is configured to receive the second stream of vital data and update the first stream of vital data by comparing the first and second stream of vital data.

Clause 8. The system of clause 6, wherein the first predetermined interval is 2 to 10 seconds and the second predetermined interval is in a range between 1 to 10 minutes.

Clause 9. The system of clause 6, wherein the first stream of vital data is down-sampled to a third predetermined interval and the second stream of vital data is up-sampled to the third predetermined interval.

Clause 10. The system of clause 1, wherein the alert generator is configured to detect a trend in the stream of inferences as a base for generating the alert.

Clause 11. The system of clause 1, wherein the MEWS is calculated from a plurality of parameters including systolic blood pressure (SBP), heart rate (HR), respiratory rate (RR), temperature (Temp), level of consciousness (AVPU), age, and body mass index (BMI).

Clause 12. The system of clause 11, wherein the MEWS is calculated by assigning a point value to each of the plurality of parameters, and the MEWS is a sum of all the point values.

Clause 13. The system of clause 1, wherein the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof.

Clause 14. The system of clause 1, wherein the trained model includes a recurrent neural network (RNN) with long-short-term-memory (LSTM) units.

Clause 15. The system of clause 1, wherein the trained model is trained with historical vital data, measurement data and modified early warning scores from multiple persons.

Clause 16. The system of clause 15, wherein the trained model is trained with training samples and labels, the training samples being built as a first and second fixed-length time sequence each containing multiple synchronized features aligned in time between the first and second sequence, the training label being built by comparing a sequence with a predetermined threshold of a modified early warning score (MEWS) and a duration of the sequence being above the predetermined threshold.

Clause 17. The system of clause 15, wherein the trained model is validated with at least one clinical deterioration event including at least one unexpected intensive care unit (ICU) transfer event, intubation event, rapid response team activation event, cardiac arrest event, or in-hospital death event.

Clause 18. A computer-implemented method, including: receiving, by a signal processing engine, a stream of vital data of a person measured controllably at a predetermined interval by at least one wearable device patched to a body of the person; receiving, by the signal processing engine, measurement data from an electronic medical record associated with the person, the measurement data being time overlapped with the stream of vital data; validating, by the signal processing engine, the stream of vital data by comparing the stream of vital data with the measurement data; generating, by at least one processor executing a trained model, a stream of inferences from the validated stream of vital data, wherein the trained model is trained with continuous monitoring data generated by a plurality of wearable devices on multiple persons; and generating, by an alert generator, an alert signal, to a monitoring system associated with the person, based at least in part on comparing the stream of inferences with a predetermined rule, the predetermined rule including a threshold of a modified early warning score (MEWS) and a duration of the stream of inferences being above the threshold, or a discrete timing of a set of hard clinical outcomes including but not limited to: cardiac arrests, intubations, rapid response team calls, unplanned ICU transfers and expirations.

Clause 19. The method of clause 18, wherein the predetermined rule includes a threshold of a modified early warning score (MEWS) and a duration of the stream of inferences being above the threshold.

Clause 20. A non-transitory computer readable medium storing program instructions that, when executed by a processor, cause a computer system to perform operations including: receiving a stream of vital data of a person measured controllably at a predetermined interval by at least one wearable device patched to a body of the person; receiving measurement data from an electronic medical record associated with the person, the measurement data being time overlapped with the stream of vital data; validating the stream of vital data by comparing the stream of vital data with the measurement data; generating, by executing a trained model, a stream of inferences from the validated stream of vital data, wherein the trained model is trained with continuous monitoring data generated by a plurality of wearable devices on multiple persons; and generating an alert signal, to a monitoring system associated with the person, based at least in part on comparing the stream of inferences with a predetermined rule, the predetermined rule including a threshold of a modified early warning score (MEWS) and a duration of the stream of inferences being above the threshold, or a discrete timing of a set of hard clinical outcomes including but not limited to: cardiac arrests, intubations, rapid response team calls, unplanned ICU transfers and expirations.

Publications cited throughout this document are hereby incorporated by reference in their entirety. While one or more embodiments of the present disclosure have been described, it may be understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be conducted in any desired order (and any desired steps may be added and/or any desired steps may be eliminated).

1. A system, configured for early-detection of an event of health condition deterioration of a subject, comprising:
at least one first wearable device patched to a body of a person for measuring at least one vital sign thereof, wherein the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof;
a controller configured to schedule the at least one first wearable device to continuously measure the at least one vital sign of the person at a first predetermined interval to generate a first stream of vital data;
a signal processing engine configured to receive the first stream of vital data from the at least one first wearable device and measurement data from an electronic medical record associated with the person, the measurement data being time overlapped with the first stream of vital data, the signal processing engine configured to validate the first stream of vital data by comparing the first stream of vital data with the measurement data;
a trained model configured to receive the validated first stream of vital data to generate a stream of inferences, wherein the trained model is trained with continuous monitoring data generated by a plurality of wearable devices on multiple persons, and comprises a recurrent neural network (RNN) with long-short-term-memory (LSTM) units; and
an alert generator configured to generate an alert signal, to a monitoring system associated with the person, based at least in part on comparing the stream of inferences to a predetermined rule, the predetermined rule comprising a threshold of a modified early warning score (MEWS) and a duration of the stream of inferences being above the threshold.

2. The system of claim 1, wherein the measurement data are generated by episodically measuring vital signs of the person using standard hospital equipment.

3. The system of claim 2, wherein the measurement data is selected from systolic blood pressure (SBP), heart rate (HR), respiratory rate (RR), temperature (Temp), oxygen saturation (SpO2), level of consciousness (AVPU) and any combination thereof.

4. The system of claim 1, wherein the signal processing engine is further configured to reject at least one artifact from the first stream of vital data for validating the first stream of vital data.

5. The system of claim 4, wherein the at least one artifact comprises data assumed of physiologically implausible values.

6. The system of claim 1, further comprising at least one second wearable device controlled by the controller to continuously measure the at least one vital sign of the person at a second predetermined interval to generate a second stream of vital data.

7. The system of claim 6, wherein the signal processing engine is configured to receive the second stream of vital data and update the first stream of vital data by comparing the first and second stream of vital data.

8. The system of claim 6, wherein the first predetermined interval is 2 to 10 seconds and the second predetermined interval is in a range between 1 to 10 minutes.

9. The system of claim 6, wherein the first stream of vital data is down-sampled to a third predetermined interval and the second stream of vital data is up-sampled to the third predetermined interval.

10. The system of claim 1, wherein the alert generator is configured to detect a trend in the stream of inferences as a base for generating the alert.

11. The system of claim 1, wherein the MEWS is calculated from a plurality of parameters including systolic blood pressure (SBP), heart rate (HR), respiratory rate (RR), temperature (Temp), level of consciousness (AVPU), age, and body mass index (BMI).

12. The system of claim 11, wherein the MEWS is calculated by assigning a point value to each of the plurality of parameters, and the MEWS is a sum of all the point values.

13. The system of claim 1, wherein the trained model is trained with historical vital data, measurement data and modified early warning scores from multiple persons.

14. The system of claim 13, wherein the trained model is trained with training samples and labels, the training samples being built as a first and second fixed-length time sequence each containing multiple synchronized features aligned in time between the first and second sequence, the training label being built by comparing a sequence with a predetermined threshold of a modified early warning score (MEWS) and a duration of the sequence being above the predetermined threshold.

15. The system of claim 13, wherein the trained model is validated with at least one clinical deterioration event comprising at least one unexpected intensive care unit (ICU) transfer event, intubation event, rapid response team activation event, cardiac arrest event, or in-hospital death event.

16. A computer-implemented method, comprising:
receiving, by a signal processing engine, a stream of vital data of a person measured controllably at a predetermined interval by at least one wearable device patched to a body of the person for measuring at least one vital sign thereof, wherein the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof;
receiving, by the signal processing engine, measurement data from an electronic medical record associated with the person, the measurement data being time overlapped with the stream of vital data;
validating, by the signal processing engine, the stream of vital data by comparing the stream of vital data with the measurement data;
generating, by at least one processor executing a trained model, a stream of inferences from the validated stream of vital data, wherein the trained model is trained with continuous monitoring data generated by a plurality of wearable devices on multiple persons, and comprises a recurrent neural network (RNN) with long-short-term-memory (LSTM) units; and
generating, by an alert generator, an alert signal, to a monitoring system associated with the person, based at least in part on comparing the stream of inferences with a predetermined rule, the predetermined rule comprising a threshold of a modified early warning score (MEWS) and a duration of the stream of inferences being above the threshold.

17. The method of claim 16, wherein the MEWS is calculated from a plurality of parameters including systolic blood pressure (SBP), heart rate (HR), respiratory rate (RR), temperature (Temp), level of consciousness (AVPU), age, and body mass index (BMI).

18. The method of claim 17, wherein the MEWS is calculated by assigning a point value to each of the plurality of parameters, and the MEWS is a sum of all the point values.

19. The method of claim 16, wherein the trained model is trained with historical vital data, measurement data and modified early warning scores from multiple persons.

20. A non-transitory computer readable medium storing program instructions that, when executed by a processor, cause a computer system to perform operations comprising:
receiving a stream of vital data of a person measured controllably at a predetermined interval by at least one wearable device patched to a body of the person for measuring at least one vital sign thereof, wherein the at least one vital sign is selected from systolic blood pressure (SBP), heart rate (HR), respiratory rate (RR), temperature (Temp), level of consciousness (AVPU), age, body mass index (BMI), oxygen saturation (SpO2), modified early warning score, person movement, and any combination thereof;
receiving measurement data from an electronic medical record associated with the person, the measurement data being time overlapped with the stream of vital data;
validating the stream of vital data by comparing the stream of vital data with the measurement data;
generating, by executing a trained model, a stream of inferences from the validated stream of vital data, wherein the trained model is trained with continuous monitoring data generated by a plurality of wearable devices on multiple persons, and comprises a recurrent neural network (RNN) with long-short-term-memory (LSTM) units; and
generating an alert signal, to a monitoring system associated with the person, based at least in part on comparing the stream of inferences with a predetermined rule, the predetermined rule comprising a threshold of a modified early warning score (MEWS) and a duration of the stream of inferences being above the threshold.
